(19) United States
(12) Patent Application Publication
(10) Pub. No.: US 2004/0191215 A1
(43) Pub. Date: Sep. 30, 2004

(54) Compositions for induction of a therapeutic response

(76) Inventors: Michael Froix, Mountain View, CA (US); Walter Bruszewski, San Francisco, CA (US)

Correspondence Address: PERKINS COIE LLP, P.O. BOX 2168, MENLO PARK, CA, 94026, US

(21) Appl. No.: 10/808,927

(22) Filed: Mar. 24, 2004

Related U.S. Application Data
(60) Provisional application No. 60/457,702, filed on Mar. 25, 2003.

(51) Int. Cl. A61K 38/19

(52) U.S. Cl. 424/85.1

(57) ABSTRACT

Compositions for attracting specific cells to an in vivo site and for stimulating the attracted cells and local resident cells to achieve a desired therapy are described. In one embodiment, a composition for initiating and promoting repair and regeneration of tissue is described. In another embodiment, a composition for inducing a cytotoxic response to tumor cells is described. The compositions are comprised of drug reservoirs containing one or more therapeutic agents effective (1) to attract one or more desired cells to the tissue site; (2) to stimulate activity, e.g., proliferation, differentiation, and/or release of biological factors that promote a desired activity, in the attracted cells; and (3) to prolong survival of the attracted cells and, if desired, local resident cells. A device for administering the composition at a desired site is also described.

This application claims the benefit of U.S. Provisional Application No. 60/457,702, filed Mar. 25, 2003, incorporated herein by reference in its entirety.

### FIELD OF THE INVENTION

The present invention relates to compositions for attracting specific cells to an in vivo site and for stimulating the attracted cells and local resident cells to achieve a desired therapy. More specifically, the invention relates to compositions useful for (1) regenerating cells and tissue in vivo at a site of damaged or injured tissue or (2) inducing a cellular immune response to cancer. Deposition of the composition at a site of damaged tissue or at a tumor site is effective to attract cells necessary, in the case of tissue repair, to regenerate cells and tissue to thereby promote quasi-normal function and blood flow in the damaged tissue region, or, in the case of cancer immunotherapy, to effect destruction of tumor tissue. The invention also provides a device for deposition of the compositions at a desired site in vivo.

### BACKGROUND OF THE INVENTION

Degeneration, necrosis, and subsequent loss of function or fibrosis, e.g., collectively herein referred to as “tissue damage” or “tissue trauma”, can result from disease or injury. For example, necrosis of myocardium and fibroblastic proliferation can result from ischemia due to coronary artery occlusion. Further, ischemia due to stroke can result in cerebral degeneration and necrosis. Tissue trauma can also be components of a variety of diseases, including diabetes, multiple sclerosis, cirrhosis, renal failure, and the like. Trauma also results from disruption of tissue by surgery or physical injury.

The body provides for mechanisms of tissue repair involving the interaction of elements of both the coagulation cascade and the immune system. The process of tissue repair is often divided into three phases: (1) inflammatory; (2) proliferative; and (3) remodeling. Although these phases are defined as distinct events, they occur as a continuum and the point at which tissue repair moves from one phase to another is subjective.

However, the endogenous tissue repair mechanism is absent in some tissue damage or inadequate to effect a full recovery of the tissue. For example, injury to axons in the central nervous system results in a permanent damage in which the neurons are unable to regenerate for restoration of tissue function. Type I diabetes, cirrhosis of the liver, congestive heart failure, skeletal muscle atrophy, Parkinson's disease, spinal cord injuries are other examples of conditions that can result in loss of tissue function. The death of vital organ tissue from either disease injury or genetic deficiency can result in total or partial loss of organ function, and often the loss of function is irreparable and irreversible.

Episodes of ischemia are another cause of considerable tissue damage. Ischemic episodes leading to tissue damage result in myocardial infarctions, stroke, skeletal muscle infarcts, and other disorders. No present treatment for these conditions offers a cure or facilitates regeneration of the traumitized, e.g., necrotic, lost or fibrosed, nonfunctional tissue.

Thus, there is a great need for mechanisms that promote regeneration of tissue. In particular, it would be desirable to provide a composition and method to reconstitute tissue that can integrate with undamaged tissue to promote blood flow in or adjacent to the damaged tissue region and to permit quasi-normal function to the tissue.

There is also a need for improved compositions and method of treating cancer. Cancer can arise in many tissues and organs in the body and often involves a primary tumor that may release cells that are distributed to metastatic sites through the blood vascular or lymphatic system. Eliminating a cancer once it has established requires that all malignant cells be destroyed or removed without killing the patient. Both primary tumors and metastases are typically treated today by surgical resection, chemotherapy, radiotherapy, or immunotherapy. Surgical treatment is frequently hindered by inaccessibility of some tumor masses within organs, such as the brain, liver, lung, and pancreas. Also, it is difficult to eradicate every cancer cell, since surgery can not remove every metastasis, and the treatments that kill cancer cells are also toxic to normal cells. If a few cancer cells remain, they can proliferate to produce a resergence of the disease and unlike normal cells they may develop resistances to the therareutic agents used against them. Thus, neither surgery, chemotherapy, nor radiation provides a lasting immunity to the recurrence of the disease.

Stimulation of the immune system to recognize and destroy cancer cells offers a less aggressive, but more effective, long term approach to treating cancer. Experiments in animals have provided evidence for immune responses to tumors and have shown that T lymphocytes are a mediator of tumor immunity. Advances in the understanding of antigen presentation and the molocules involved in T-cell activation have provided new immunotherapy strategies based on a better understanding of the immune response. While immunotherapy of cancer has shown promising results in a number of animal tumor systems, clinical trials of immunotherapy have produced inconsistently successful results (Janeway, C. et al., Immunobiology: The Immune System in Health and Disease, 5th Edition, Garland Science Publishing, 2001).

Tumor infiltrating lymphocytes (TIL) have generally been considered to be agents of cytotoxic attack by an immunocompetent host on tumor cells presenting recognizable tumor antigens. Tumor cells have, however, evolved a number of strategies to prevent immunological recognition by the host. Thus, there is a need for mechanisms that promote, at the site of the primary malignant tumor or metastasis, tumor antigen recognition and development of a cytotoxic response to the tumor cells. There is also a need to promote infiltration of tumors by cytotoxic cells. There is a particular need to promote these phenomena in tumor masses in remote sites that are not treatable by surgery or radiotherapy.

### SUMMARY OF THE INVENTION

In one aspect, the invention includes a composition for inducing a cellular response, comprising a first agent effective to attract one or more desired cells to a tissue site; a second agent effective to stimulate activity of such cells; and a third agent effective to influence survival of such cells. In one embodiment, composition is for promoting regeneration of cells and/or tissue and said first agent is effective to attract one or more of stem cells, progenitor cells, and accessory cells to the tissue site. In another embodiment, the composition is for inducing an immune response to a tumor and the first agent is effective to attract one or more cells selected from the group consisting of T-lymphocytes, macrophages, polymorphonuclear leucocytes, antigen-presenting cells, and natural killer cells.

In one embodiment, second agent is effective to stimulate an activity selected from proliferation and differentiation.

The tissue for induction of the therapeutic response, in various embodiments, is skeletal muscle, liver, pancreas, brain, cardiac muscle, central nervous system, or tumor tissue.

In one embodiment, the tissue is cardiac tissue and the first agent attracts cells selected from the group consisting of circulating blood monocytes, circulating angiogenic cells, and circulating arteriogenic cells; the second agent stimulates release from said cells of factors to promote angiogenesis and/or arteriogenesis; and the third agent influences the survival of circulating blood monocyte-derived macrophages resident in the cardiac tissue.

The first agent, in one embodiment, is selected from the group consisting of macrophage chemoattractant protein (MCP)-1, MCP-2, MCP-3, MCP-4, MCP-5, regulated upon activation, normal T-cell expressed and secreted cytokine (RANTES), Fraktalkines, macrophage inflammatory protein (MIP)-1-alpha, MIP-1-beta, N-farnesyl peptides, complement activation product C5a, leukotriene B4, platelet activating factor (PAF), transforming growth factor beta (TGF-beta), interleukins, granulocyte macrophage colony-stimulating factor (GM-CSF), granulocyte colony-stimulating factor (G-CSF), colony stimulating factor-1 (CSF-1), and macrophage colony-stimulating factor (M-CSF).

The second agent, in another embodiment, is selected from the group consisting of macrophage chemoattractant protein (MCP)-1, MCP-2, MCP-3, MCP-4, MCP-5, tumor necrosis factor (TNF)-α, TNF-β, regulated upon activation, normal T-cell expressed and secreted cytokine (RANTES), Fraktalkines, macrophage inflammatory protein (MIP)-1-alpha, MIP-1-beta, N-farnesyl peptides, complement activating product C5a, leukotriene B4, platelet activating factor (PAF), transforming growth factor beta (TGF-beta), interleukins, granulocyte macrophage colony-stimulating factor (GM-CSF), granulocyte colony-stimulating factor (G-CSF), colony stimulating factor-1 (CSF-1), macrophage colony-stimulating factor (M-CSF), and lipopolysaccharide.

The third agent, in other embodiments, is selected from the group consisting of GM-CSF, G-CSF, CSF-1, and M-CSF.

In another embodiment, the composition is designed to induce a therapeutic response in a tumor. In this embodiment, the first agent is selected from the group consisting of IL-8, MIG, IL-12, MCP-1, -2, -3, -4, -5, MIP-1 alpha, MIP-1 beta, MIP-1 gamma, and RANTES. The second agent is selected from the group consisting of GM-CSF and IL-12. The third agent is selected from the group consisting of MIG, platelet factor 4, MCP-1, -2, -3, and MIP-1 gamma.

The agents are released from the composition simultaneously or, in another embodiment, sequentially. The drug reservoirs in which the various agents are contained can be formed from a polymer, including biodegradable and non-biodegradable. In other embodiments, the drug reservoirs include a targeting ligand, such as a cellular adhesion molecule, associated with the external surface of the reservoirs.

The drug reservoir composition can be formulated into a dosage form selected from, for example, an emulsion, a gel, a paste, and a liquid.

In another aspect, the invention includes a method for inducing a therapeutic response at a specific tissue site by depositing in or adjacent to a selected tissue site a composition, described above, comprised of one or more drug reservoirs containing one or more therapeutic agents effective to induce the desired response. In brief, the drug reservoirs include one or more agents effective to (i) attract to the tissue site one or more of stem cells selected from the group consisting of progenitor cells, accessory cells, T-lymphocytes, macrophages, polymorphonuclear leucocytes, antigen-presenting cells, and natural killer cells; (ii) stimulate the one or more attracted cells to undergo an activity selected from proliferation and differentiation; and (iii) influence the survival of the one or more attracted cells in the tissue.

In one embodiment, the tissue is cardiac tissue and said depositing is effective to promote arteriogenesis and/or angiogenesis.

In another embodiment, the tissue is a tumor mass and said depositing is effective to attract cytotoxic cells into the tumor.

In another aspect, the invention includes a method for promoting cellular and/or tissue regeneration. The method includes depositing in or adjacent to a selected tissue site one or more drug reservoirs containing one or more therapeutic agents effective to (i) attract one or more of stem cells, progenitor cells, and accessory cells to the tissue site; (ii) stimulate direct action on local and attracted cells and components of the extracellular matrix, and the release of biological agents from local and attracted cells that promote cellular and/or tissue regeneration; and (iii) influence the survival of the attracted and locally regenerated cells in such tissue.

In one embodiment, the reservoirs deposited at the tissue site contain one or more biological agents.

In another embodiment, the composition is administered for regeneration of damaged parenchymal tissue. An exemplary tissue is myocardial tissue, where, for example, arteriogenesis and/or angiogenesis are promoted.

The biological agents contained in the drug reservoirs include, in an exemplary embodiment for regeneration of the myocardium, (i) a first agent effective to attract cells selected from circulating blood monocytes, circulating angiogenic cells, and circulating arteriogenic cells; (ii) a second agent effective to stimulate release of factors to promote angiogenesis and/or arteriogenesis; and (iii) a third agent effective to influence the survival of circulating blood monocyte-derived macrophages resident in the myocardial tissue.

The first agent, in one embodiment, is a peptide effective to attract progenitor cells, stem cells, and, optionally, accessory cells. The first agent can be macrophage chemoattractant protein (MCP)-1, MCP-2, MCP-3, MCP-4, MCP-5, regulated upon activation, normal T-cell expressed and secreted cytokine (RANTES), Fraktalkines, macrophage inflammatory protein (MIP)-1-alpha, MIP-1-beta, N-farnesyl peptides, complement activation product C5a, leukotriene B4, platelet activating factor (PAF), transforming growth factor beta (TGF-beta), interleukins, granulocyte macrophage colony-stimulating factor (GM-CSF), granulocyte colony-stimulating factor (G-CSF), colony stimulating factor-1 (CSF-1), or macrophage colony-stimulating factor (M-CSF), and functionally equivalent fragments thereof.

The second agent, in one embodiment, is effective to stimulate the attracted cells and/or the locally regenerated cells. Exemplary second agents include macrophage chemoattractant protein (MCP)-1, MCP-2, MCP-3, MCP-4, MCP-5, tumor necrosis factor (TNF)-α, TNF-β, regulated upon activation, normal T-cell expressed and secreted cytokine (RANTES), Fraktalkines, macrophage inflammatory protein (MIP)-1-alpha, MIP-1-beta, N-farnesyl peptides, C5a, leukotriene B4, platelet activating factor (PAF), transforming growth factor beta (TGF-beta), interleukins, granulocyte macrophage colony-stimulating factor (GM-CSF), granulocyte colony-stimulating factor (G-CSF), colony stimulating factor-1 (CSF-1), macrophage colony-stimulating factor (M-CSF), lipopolysaccharide, and functionally equivalent fragments thereof.

The third biological agent, in one embodiment, is selected from the group consisting of GM-CSF, G-CSF, CSF-1, M-CSF, and functionally equivalent fragments thereof.

Other exemplary biological agent for including in the drug reservoirs include fibroblast growth factor (FGF, FGF-1, FGF-2), TGF-alpha, insulin-like growth factor (IGF-1), angiopoietin-1, angiopoietin-2, vascular endothelial growth factor (VEGF), constructs of VEGF such as VEGF-2, VEGF165, and VEGF121, platelet derived growth factor (PDGF)-A, PDGF-B, PDGF-BB, endothelial mitogenic growth factors, and functionally equivalent fragments thereof.

Tissues contemplated for treatment using the method include, for example, skeletal muscle, liver, pancreas, brain, cardiac muscle, and central nervous system.

In one embodiment, the drug reservoirs have a biological ligand attached to the external surface of the drug reservoirs. Exemplary ligands include cellular adhesion molecules.

In another embodiment, the drug reservoirs are formulated from a biodegradable polymer or from a non-biodegradable polymer. In another embodiment, the drug reservoirs are comprised of a vesicle-forming lipid or are formulated into a dosage form selected from an emulsion, a gel, a paste, and a liquid.

The drug reservoirs can be, in some embodiments, deposited into an interstitial tissue space, where they have mobility.

In yet another aspect, the invention includes a composition for promoting regeneration of cells and/or tissue, comprising a first agent effective to attract one or more of stem cells, progenitor cells, and accessory cells to said tissue; a second agent effective to stimulate activity of such cells and/or of locally regenerated cells; and a third agent effective to influence survival of such cells.

In one embodiment, the agents are released from the composition simultaneously. In another embodiment, the agents are released from the composition sequentially.

In yet another embodiment, the agents are packaged in a spherical drug reservoir. The drug reservoir can have, in some embodiments, a biological ligand attached to the external surface. An exemplary ligand is a cellular adhesion molecule.

In still another aspect, the invention includes a composition for attracting immune cells to a site to promote the destruction of cancerous cells. The method includes depositing in or adjacent to a selected site one or more drug reservoirs containing one or more therapeutic agents effective to (i) attract one or more of lymphocytes, macrophages, antigen presenting cells (including dendritic cells), inflammatory cells, natural killer cells, progenitor cells, and accessory cells to the tissue site; (ii) stimulate direct action on local and attracted cells and components of the extracellular matrix, and the release of biological agents from said local and attracted cells that promote cytotoxicity to cancerous cells; and (iii) influence the survival of the attracted and locally regenerated cells in such tissue and to promote an immunogenic response to the cancerous cells.

In an exemplary embodiment, reservoirs contain biological agents which can accomplish the following functions: (i) chemoattraction of lymphocytes, macrophages, antigen presenting cells (including dendritic cells), and inflammatory cells, including polymoronuclear leucocytes from the blood stream, lymphatic vessels and adjacent tissue into the central region of the tumor mass; (ii) stimulation of tumor antigen presentation to lymphocytes by antigen presenting cells, culminating in development of a systemic cellular immune response (in the form of cytotoxic lymphocytes and natural killer cells) directed against all tumor cells in the body, including those in the tumor receiving the composition and in other tumor masses in the body; and (iii) enhancing the survival of the attracted and local resident cells, to promote an immunogenic response to the cancerous cells, including promoting trafficking of cytotoxic and natural killer TIL into the tumor mass resulting in tumor cell destruction.

In one embodiment, chemoattraction of cells is achieved by including in the drug reservoirs one or more chemoattractive agents selected from the group consisting of IL-8, MIG, IL-12, MCP-1, -2, -3, 4, -5, MIP-1 alpha, MIP-1 beta, MIP-1 gamma, and RANTES.

In another embodiment, the drug reservoirs include a stiumlation agent selected from the group consisting of GM-CSF and IL-12.

In yet another embodiment, the third agent in the drug reservoirs for promoting survival of the attracted cells and for promoting an immunotherapeutic response to the tumor is one or more agents selected from MIG, platelet factor 4, MCP-1, -2, -3, and MIP-1 gamma.

The invention also includes, in another aspect, a device for delivery of a composition, and in particular, a composition as described above. The device is comprised of a catheter shaft having a lumen extending therethrough and terminating in a port, a distal region being at least partly flexible, a proximal end being at least partly rigid; a monorail segment positioned adjacent to the distal end of the catheter shaft and having a guidewire engagement segment; a user interface with a distal end and a proximal end being rigidly connected at the interface distal end to the proximal end of the catheter shaft; and a sheath connected to the proximal end of the interface for housing a movable piston.

In one embodiment, the device may further include a needle positioned at least partly in the catheter shaft in a non-deployed state and deployable from the catheter shaft in a deployed state. The needle may be straight or curved.

In another embodiment, the guidewire engagement segment further includes an endoluminal paving device for deposition of the composition along a wall of a vessel or a body cavity. In this embodiment, the endoluminal paving device has an elongate, flexible cup with a central cavity for receiving the composition from the catheter shaft port. Further, in this embodiment, the catheter shaft port is positioned adjacent to the endoluminal paving device and delivers the composition into the endoluminal paving device. The endoluminal paving device may include at least an upper opening and a lower opening for introduction of the guidewire through the cup. Preferably, a helical guidewire is used with the endoluminal paving device.

The device may also include a composite tube extending at least partly through the shaft distal region. The composite tube may be comprised of a thermoplastic elastomer and wire braid. In one embodiment, the composite tube contacts the proximal end of the needle. In another embodiment, the composite tube is adhered to the proximal end of the needle. The proximal end of the catheter shaft may further include a reinforced shaft segment contacting the distal end of the user interface.

The device may further include at least one hypotube extending at least partly through the shaft proximal region. In one embodiment, the device includes at least two hypotubes to lend structural support or rigidity to the proximal region of the catheter shaft.

The distal region and the proximal region may be joined at a transition region. In one embodiment, the transition region is formed of a fused, thermoformed, thermoplastic elastomer spanning the proximal and distal regions. In another embodiment, the transition region includes an adaptor joined to a distal end of the composite tube and a proximal end of the at least one hypotube.

The drug delivery device may further include at least one balloon positioned on a distal portion of the monorail segment. In one embodiment, the at least one balloon comprises at least two balloons positioned on opposing sides of the monorail segment. In another embodiment, at least one of the at least one balloon has a biconical profile when inflated. The device may further include one or more passages in catheter shaft for introduction and removal of fluid, especially to and from the balloons.

In yet another aspect, the invention includes a delivery device for delivery of a composition comprising (i) a catheter shaft having a lumen extending therethrough to a port, a distal region being at least partly flexible, a proximal end being at least partly rigid, a transition region positioned between the distal and proximal regions, and a reinforced shaft segment at the proximal end, (ii) a monorail segment positioned adjacent to the distal end of the catheter shaft and having a guidewire engagement segment, (iii) a user interface with a distal end and a proximal end being rigidly connected at the interface distal end to the proximal end of the catheter shaft, and (iv) a sheath connected to the proximal end of the interface for housing a movable piston. The device includes a composite tube extending at least partly through proximal shaft region and terminating at the transition region. The device includes at least one hypotube extending at least partly through the proximal region of the catheter shaft.

These and other objects and features of the invention will be more fully appreciated when the following detailed description of the invention is read in conjunction with the accompanying drawings.

### BRIEF DESCRIPTION OF THE DRAWINGS

FIG. 1 is an illustration of the overall view of tissue regeneration processes at the cellular level resulting from deposition of a composition in accord with the invention;

FIG. 2 is an oblique detail view of the surface and cross section of an exemplary drug reservoir for use in the composition;

FIG. 3 is a plan view of an exemplary drug reservoir depositing device;

FIG. 4 is a detail plan view of the catheter shaft of the device in FIG. 3;

FIGS. 5A-5C are detail views of a distal segment of the catheter shaft showing guidewire engagement in the monorail segment (FIG. 5A) and needle tip for tissue penetration (FIGS. 5B, 5C);

FIGS. 6A-6B are transverse cross-sectional views through lines 6A-6A and 6B-6B in FIG. 5C;

FIGS. 7A-7B show balloon-catheter embodiments having a biconical balloon profile (FIG. 7A) and having two spherical balloons (FIG. 7B);

FIGS. 7C-7D are views of balloon-catheter embodiments showing positioning of the biconical balloon (FIG. 7C) and the double spherical balloon (FIG. 7D) that affords needle penetration of tissue in blood vessels;

FIGS. 8A-8B are cross-sectional views of region A in FIG. 4 with the needle retracted (FIG. 8A) and extended from the catheter shaft (FIG. 8B);

FIG. 9A is a cross-sectional view of region B in FIG. 4;

FIG. 9B is a cross-sectional view through line F-F in FIG. 9A;

FIGS. 9C-9D are cross-sectional views similar to FIG. 9B, but in devices with a biconical balloon (described in FIG. 7A) or with two spherical balloons (described in FIG. 7B);

FIG. 10 is a longitudinal cross-section of Area D in FIG. 4;

FIG. 11 is a longitudinal cross-section of Area C in FIG. 4;

FIG. 12 is a longitudinal cross-section of Area E in FIG. 4;

FIG. 13 illustrates use of the device for deposition of a bolus of drug reservoir formulation into the myocardium at an ischemic site;

FIGS. 14A-14C show an alternative embodiment where a helical guidewire is used for advancing the device and positioning the needle for tissue penetration;

FIG. 15 is an overall top view of the user interface of the device;

FIGS. 16A and 16B are side (FIG. 16A) and oblique (FIG. 16B) views of the user interface shown in FIG. 15;

FIGS. 17A-17B are oblique views of the user interface showing the proximal segment in its withdrawn position (FIG. 17A) and in its withdrawn position and rotated 90°; and

FIGS. 18A-18D are various views of an endoluminal paving device for deposition of the composition along the wall of a vessel or cavity in the body.

### DETAILED DESCRIPTION OF THE INVENTION

### I. DEFINITIONS

“Tissue” as used herein intends a whole organ, either in vivo or ex vivo, a fragment of an organ, or two or more cells.

“Parenchyma” as used herein refers to the specific functional tissue which distinguishes a particular organ. For example, the parenchyma of the heart is the myocardium, consisting of cardiac muscle fibers and their blood vascular system. Further, the parenchyma of the liver consists of mainly hepatocytes.

“Degeneration” refers to reversible changes in cells and tissues in response to a variety of insults. These insults include exposure to noxious agents (both chemical and biological) and physical factors, which include pressure and extremes in heat and cold. Prolonged or intensified exposure to such insults can result in cell death and necrosis.

“Necrosis” refers to morphological changes in cells that follow cell death. “Necrotic tissues” thus comprise groups of cells demonstrating necrotic morphology.

“Tissue damage” as used herein refers to one or more effects of various insults on tissue; these effects are to include degeneration of parenchymal cells, cell death, necrosis, tissue loss, and fibroblastic or glial scarring.

“Stem cell” as used herein generally refers to a special type of cell that has a unique capacity to renew itself and to give rise to specialized cell types. Although most cells of the body, such as heart cells or skin cells, are committed to conduct a specific function, a stem cell is uncommitted and remains uncommitted until it receives a signal to develop into a specialized cell. Stem cells can make identical copies of themselves and they can also give rise to mature cell types that have characteristics morphologies and specialized functions. Typically stem cells generate an intermediate cell type or types before they achieve their fully differentiated state. The intermediate cell is called a precursor or progenitor cell. Stem cells can renew themselves mitotically, and can also (by mitosis) give rise to progenitor cells that are capable of differentiation into cellular components of quasi-functional tissues found in adult individuals. Stem cells as used herein include adult stem cells which are undifferentiated cells distributed throughout the body of an adult individual in a variety of differentiated tissues, including peripheral blood, blood and bone marrow derived hematopoietic, stromal and mesenchymal stem cells. Hematopoietic stem cells existing in adult bone marrow for example can populate the cerebral cortex with highly differentiated Purkinje cell neurons, which are central to the function of normal cortical neural circuits (Wagers et al., Science, 297: 2256 (2002)).

As used herein, a “progenitor cell” intends a cell capable of participating in the process of the regeneration of healthy tissue. “Progenitor cells” are among the daughter cells from mitosis of stem cells. These cells are distinguished from stem cells by commitment to a differentiation program (stimulated by a variety of agents) that produces partially differentiated families of cell classes. These cell classes ultimately regenerate a specific tissue with cells of a fully differentiated and specialized phenotype normally found in the parenchyma of that tissue. For example, cardiac myocytes, skeletal myoblasts, alpha, beta, and delta cells of the pancreas, hepatocytes, neurons, astroglia, oligodendroglia, and microglia of the central nervous system may all descend from bone marrow stem cells and adult stem cells of local tissue origin and remain in the differentiated tissue of the particular organ. This list is merely exemplary and non-limiting, but each of these cells have the capability of initiating tissue regeneration. Further examples of progenitor cells are partially differentiated cells within the adult brain that are daughters of neural adult stem cells that reside in specific regions (e.g., the dentate gyrus of the hippocampus and the subventricular zone). These partially differentiated cells migrate to distant regions of the brain. These cells are committed to become either fully differentiated neurons, or specific types of glial cells, at these distant sites. The neurons take on normal functions within the neural circuitry. The glial cells perform differentiated functions characteristic of glial type. For example, oligodendroglial cells carry out a program of creating myelin, an essential extracellular matrix component in the CNS which electrically insulates the axons of neurons. Satellite cells are progenitors of skeletal muscle fibers and typically reside near the surface of differentiated muscle fibers. Satellite cells enter mitosis and fuse to form differentiated, multinucleated muscle fibers. Progenitor cells can be attracted to a particular tissue region of interest by the presence of an appropriate chemoattractant to begin the process of regeneration of healthy tissue.

“Accessory cell” as used herein refers to a cell that is involved in the regeneration of parenchymal cells, but that is not a parenchymal cell, stem cell, or parenchymal cell progenitor; the accessory cell synthesizes and secretes biological factors that stimulate stem cells and progenitor cells (for example, bone marrow stromal cells, functioning as accessory cells, produce hepatocyte growth factor (HGF), which prevents neuron apoptosis, and nerve growth factor (NGF), neurotropin 4 (NT4), and brain-derived neurotrophic factor (BDNF), all of which stimulate proliferation and differentiation of neuronal progenitors, and suppress apoptosis of differentiated neurons; the accessory cell synthesizes and secretes extracellular matrix components essential to the functional architecture of the parenchymal tissue; the accessory cell synthesizes and secretes biological agents which modify and remodel the extracellular matrix to facilitate parenchymal regeneration from damaged tissue; examples of accessory cells include but are not limited to the following: (a) tissue macrophages derived from circulating blood monocytes; (b) bone marrow stromal cells which give rise to mesenchymal cells in regenerating tissues; (c) microglia resident in the central nervous system.

“Stimulate” or “stimulate an attracted cell or a locally regenerated cell” as used herein refer to induction of a wide variety of activities and phenomena associated with cells that are targets of a stimulant. These activities and phenomena include:

(i) increased motility within a tissue;

(ii) increased motility from one tissue to another (e.g., release of stem cells from the bone marrow into the blood stream, and subsequently, into a distant tissue);

(iii) attachment of cells to other cells, to extracellular matrix, and to cellular adhesion ligands;

(iv) stimulation of DNA replication and mitosis;

(v) stimulation of synthesis and secretion of cytokines;

(vi) stimulation of synthesis and secretion of chemokines;

(vii) stimulation of synthesis and secretion of extracellular matrix components;

(viii) suppression of apoptosis;

(ix) protection from effects of degenerative agents (e.g., antioxidant effects);

(x) stimulation of synthesis and secretion of factors which degrade extracellular matrix (e.g., stimulation of release of matrix metalloproteinases by tissue macrophages to create spaces within the extracellular matrix for regenerated arterioles in response to ischemic insults and for the translocation of cells);

(xi) induction of plasticity of architecture of intercellular connection amongst differentiated cells; for example, stromal cell-derived factor 1-alpha (SDF-1α) and interleukin-6 (IL-6) are known to act directly upon differentiated CNS neurons to cause rearrangement of synaptic connections in a regenerative response to toxic damage to neurons;

(xii) promotion of differentiation of progenitors and stem cells to fully functional parenchymal cells;

(xiii) modulation and orchestration of differentiation programs of various distinct cell types within regenerated parenchyma;

(xiv) release of biological agents from attracted cells that promote angiogenesis and/or arteriogenesis;

(xv) promotion of proliferation of stem cells, progenitor cells, accessory cells, or locally regenerated cells; and/or

(xvi) stimulation of synthesis and secretion of factors, cytokines which increase the cytotoxic response of lymphocytes, B cells, dendritic cells, natural killer cells, lymphokine activated killer cells and macrophages to cancerous cells.

The term “placed at” and the term “depositing” as used in conjunction with a composition being “placed at” or “deposited” at particular tissue regions intends the introduction of the composition at or near the desired site. The composition can be placed directly at the target site within the tissue, or can be placed in a body cavity adjacent to the target tissue site. For example, placement at a tissue site in the heart will include depositing a composition within the pericardial cavity (the space between the parietal and visceral pericardium which envelopes the heart) as well as deposition of the composition within or on the heart tissue (myocardium) itself.

An agent has a “direct” effect on a cells when it acts not only by stimulating release of active substances from the cell but also by inducing mitogenesis of progenitors, differentiation of progenitors, coordination of differentiation programs of different cells classes, or the remodeling of extracellular matrix.

“Angiogenesis” as used herein refers to formation of endothelial cells leading to formation of new capillary networks.

“Arteriogenesis” as used herein refers to in situ growth of arteries by proliferation of endothelial and smooth muscle cells from preexisting arteriolar connections supplying blood to ischemic tissue, tumor, or sites of inflammation.

“Ischemic tissue” “tissue at risk of ischemia” intends a tissue, tumor, or site of inflammation experiencing an insufficient supply of blood or at risk of the same.

“Ischemia” or an “ischemic event” refers to an insufficient supply of blood to a specific cell, tissue or organ. A consequence of decreased blood supply is an inadequate supply of oxygen to the organ or tissue (hypoxia). Prolonged hypoxia may result in injury to the affected organ or tissue.

“Anoxia” refers to a virtually complete absence of oxygen in the organ or tissue, which, if prolonged, may result in death of the cell, organ or tissue.

“Hypoxia” or a “hypoxic condition” intend a condition under which a cell, organ or tissue receive an inadequate supply of oxygen.

### II. COMPOSITIONS FOR TISSUE REGENERATION AND FOR CANCER IMMUNOTHERAPY

In one aspect, the invention includes a composition suitable for in vivo administration to a tissue site that has sustained cellular-degeneration, or cell and tissue death, or necosis, tissue loss, or post-necrotic fibroblastic or glial scarring, or all of these effects, or is at risk of any one, any two or more, or all of these effects. More simply stated, the composition is suitable for use in a tissue that is post-trauma or is at risk of trauma. The composition is comprised of a first agent effective to attract at least one of a stem cell and a progenitor cell to the region of tissue trauma; a second agent effective to stimulate activity of the attracted cells; and a third agent effective to influence survival of the attracted cells, and optionally to influence survival of differentiated cells that arise from the attracted cells. The first agent in some embodiments will additionally attract accessory cells. As will be further described below, the first, second, and third agents depend on tissue type and the stem cells or proginator cells, and optional accessory cells, attracted to the site. All of the above-mentioned cells are of autologous origin. All cells are translocated to the tissue site in question via conduits normally existing in the body, including the blood and lymphatic vascular system, the cerebrospinal fluid system, and pathways of migration through interstitial spaces in the tissues of the body.

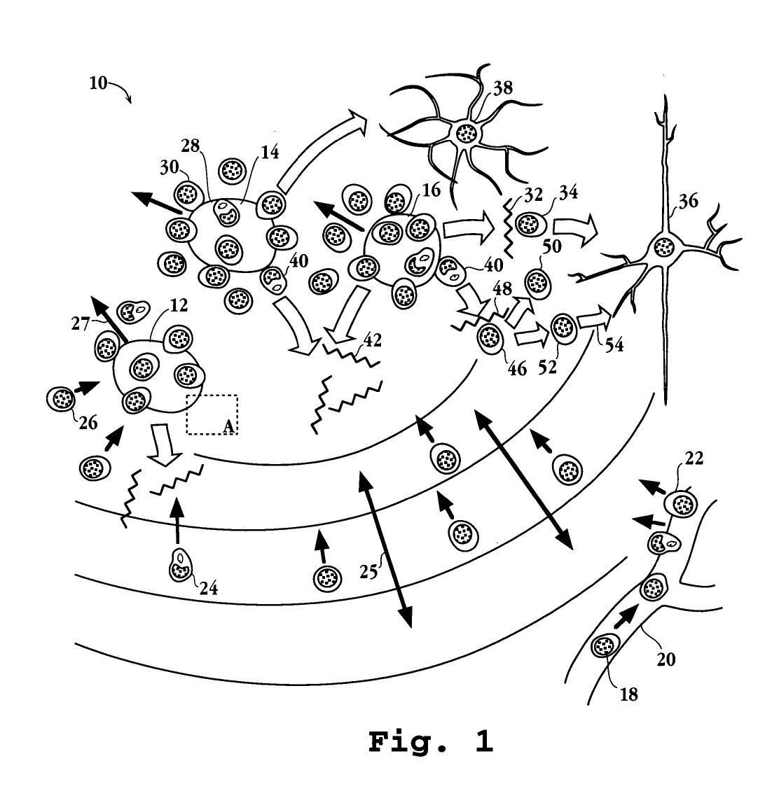

As indicated above, the composition finds use in a wide variety of tissues post-trauma or at risk of trauma, and examples of compositions tailored for specific tissues are provided below. For purposes of a general, but detailed, description of the composition, an example of a composition deposited at a site in brain tissue is provided with respect to FIG. 1. However, as will be clear from the disclosure below, the composition and process detailed in FIG. 1 is exemplary of tissue trauma in the brain and the broad, general concepts are applicable to any damaged tissue.

FIG. 1 illustrates a simplified tissue regeneration process at the cellular level at a tissue site in the brain following deposition of the claimed composition. The damaged brain tissue, indicated generally at 10, may be a result of an ischemic event, such as stroke, or a disorder, such as Alzheimers Disease, multiple sclerosis, meningoencephalitis, or due to a physical trauma. A composition is deposited at the tissue site 10, or adjacent the site, to promote regeneration of the damaged or dead tissue. In this embodiment, the composition takes the form of drug reservoirs, such as reservoirs 12, 14, 16, that are designed to contain and release selected therapeutic agents. As noted above, the drug reservoirs are prepared to contain a first therapeutic agent effective to attract stem cells and/or progenitor cells, and, optionally, accessory cells, to the tissue site; a second therapeutic agent effective to stimulate activity of the attracted cells; and a third therapeutic agent effective to influence survival of the attracted cells and, optionally, the regenerated, fully differentiated cells. In some embodiments, as will be further described below, the same agent serves as both a chemoattractant, e.g., serves as the first therapeutic agent, and as a stimulating agent and/or a survival enhancing agent. That is, in some cases, a single agent may provide one or more of the functions of the first, second, and third agents.

In this particular illustration, the drug reservoirs 12, 14, 16, contain a first agent that acts as a chemoattractant for stem cells, such as circulating bone marrow-derived stem cells, such as cell 18, in a nearby capillary 20. In response to the chemokine gradient established by release of the chemoattractant from the drug reservoirs, stem cells transmigrate through the capillary endothelium into the adjacent tissue, as illustrated by cell 22. Attracted cells can include stem cells and/or progenitor cells, such as monocytes and other cells that are capable of assisting in tissue regeneration. Accessory cells in the vicinity, such as macrophages as exemplified by macrophage 24, are attracted to the tissue region in response to the chemokine gradient, indicated at 25. Additionally, stem cells in adjacent tissue or from specific stem cell origin sites migrate to the tissue site in response to the gradient. For example, with respect to the brain, adult neural stem cells, such as cell 26, from either the dentate gyrus of the hippocampus or from the subventricular zone, are drawn to the area. The overall result of attraction of cells to the reservoirs by chemokines is the increased concentration of stem cells and/or progenitor cells, and in some cases of accessory cells, in the vicinity of the reservoir deposit, as illustrated in FIG. 1.

In some embodiments, the drug reservoirs in the composition include surface ligands for interaction with cells drawn to or present in the tissue site. This feature is illustrated in FIG. 1 by stem cells, such as cells 28, 30, attached to the cell surface of reservoir 14. The surface-attached ligand is multi-functional and can serve to provide attachment sites for the cells attracted to the damaged tissue site, to present stimulant biological factors to cell-surface receptors in a functionally effective manner, and/or to protect biological factors from degradation. The ligand is selected based on the tissue being treated and on the requirements of the attracted cells. Exemplary ligands include cell adhesion molecules, extracellular matrix components, and fragments thereof. Other exemplary ligands for particular tissues are provided below.

Drug reservoirs in the composition deposited at the tissue site contain for release an agent effective to stimulate the attracted cells into action. FIG. 1 shows a cytokine 32 that has been released from drug reservoir 16 inducing stem cell 34 to differentiate into a pyramidal neuron 36. Stem cells attached to reservoir 14 are induced by suitable stimulation agents in reservoir 14 to differentiate into a glial cell (astrocyte) 38. Additionally, macrophages attracted to the area are stimulated to produce chemokines and cytokines, such as macrophage 40 producing cytokine 42. Other cells are induced to proliferate in the presence of the stimulation agent, such as neural stem cell 46 proliferating in response to stimulation by cytokine 48 into daughter cells 50, 52. The daughter cells can further differentiate into, for example, pyramidal neurons, as indicated by arrow 54.

While not illustrated in FIG. 1, the drug reservoirs also include an agent effective to permit survival of the attracted cells or to prolong the average lifetime of attracted cells and of regenerated, fully differentiated cells in damaged tissue. Suitable agents vary according to the tissue type and the attracted cells, and examples are given below for a variety of tissues.

According to another feature of the composition, the drug reservoirs are capable of movement and dispersion from the original deposition site through the tissue space. This feature is illustrated in FIG. 1 by arrow 27 indicating movement of reservoir 12. Typically, such movement will occur as a result of concentration profiles, buoyancy, and/or fluid movement in the tissue.

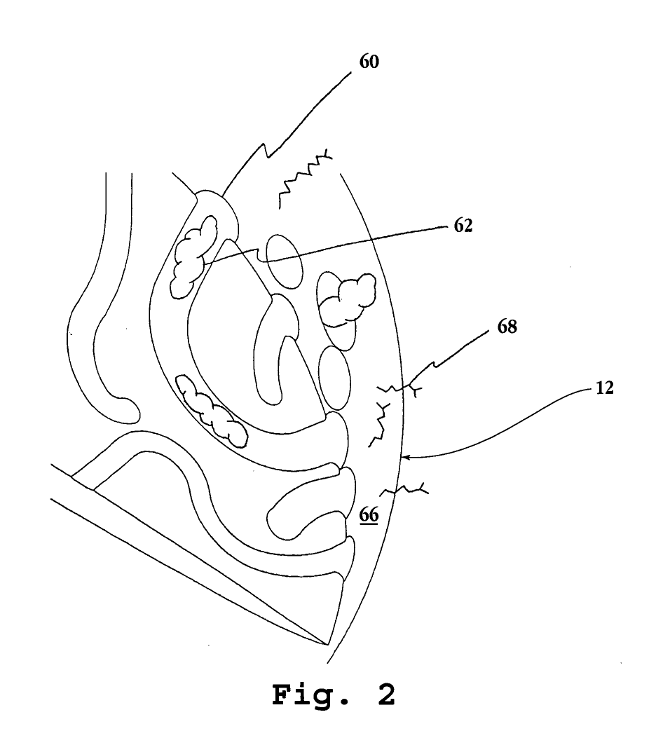

FIG. 2 is a detail view of area A in drug reservoir 12 in FIG. 1. In this embodiment, reservoir 12 is porous, as exemplified by pore opening 60. As will be further described below, other embodiments of the composition are contemplated that do not involve porous reservoirs for containment of the therapeutic agents. The therapeutic agents are contained within the pores of the reservoir, such as cytokine 62 in pore 60, but may also be held within the regions between the pores, e.g., the solid region, of the reservoir, for example, in the intermolecular spaces of the reservoir composition. Cell adhesion ligands, such as cell adhesion peptide 68, can be incorporated into the reservoir matrix so that some of these peptides are exposed on the reservoir surface, indicated at 66, in order to retain the attracted cells in close proximity with the drug reservoirs. Other embodiments of the reservoirs are contemplated in which the reservoir surface is coated with a composition which includes cell adhesion peptides which will be exposed to binding to cell-surface receptors.

In summary, deposition of the composition containing one or more therapeutic agents effective to (i) attract stem cells and/or progenitor cells, and, optionally, accessory cells, to a tissue region post-trauma or at risk of trauma, (ii) stimulate the attracted cells, e.g, the stem cells, progenitor cells, and/or accessory cells, and/or locally regenerated cells to initiate or engage the cascade of events resulting in tissue regeneration, and (iii) promote the survival of the attracted cells and other cells in the region for prolonged activity and longevity of the regenerated tissue. Regeneration of functional tissue permits quasi-normal function of the tissue, and in a preferred embodiment, the regenerated tissue will integrate with undamaged, healthy tissue adjacent the region of trauma. The composition promotes regeneration of tissue by repopulating the region of scarred and damaged tissue with viable, healthy tissue cells.

As can be appreciated from the above, the composition is broadly intended for use in regeneration of damaged tissue, and particularly damaged parenchymal tissue. The invention will now be further illustrated by way of specific compositions tailored for regeneration of specific tissues damaged by disease or trauma, where exemplary specific tissues include cardiac muscle, skeletal muscle, liver, pancreas, central nervous system, and kidney.

A. Compositions for Promoting Parenchymal Regeneration In Post-Ischemic Tissue or Tissue at Risk of Ischemia

In one embodiment, the composition is comprised of drug reservoirs containing one or more selected therapeutic agents for regeneration of tissue damaged due to an ischemic event. Treatment of disease conditions resulting from ischemia continues to be an actively explored area of medicine, particularly for use in the Western world where ischemic heart disease remains a leading cause of mortality. Cardiac ischemia arises from a sudden obstruction of a major coronary artery which leads to death of cardiac muscle cells (cardiomyocytes) and vascular structures in the supplied region of the heart. Similarly, the blockage of major vessels to other organs and tissue leads to the death of vital cells of that tissue and a severe deterioration in the ability of the tissue to maintain its normal and proper function. Cerebral ischemia, for example, where arterial blood flow to a portion of the brain is obstructed or reduced below a critical level can result in both transient ischemic attacks and stroke. While cardiac and cerebral ischemia are two of the more common forms of ischemia, it is also implicated in damage to other body tissues, such as the eye, kidney, liver, body lower limbs, the bowel, skin and in the rejection of skin flaps, etc.

Recent approaches for managing ischemia have involved attempts to stimulate the growth of new capillary networks and the development of collateral vessels via angiogenesis and arteriogenesis. Angiogenesis typically refers to the formation of new endothelial-lined vessels forming capillary networks. Arteriogenesis is distinct from angiogenesis and refers to the growth and remodeling of preexistent collateral arterioles into functional arteries, by mitosis of endothelial and smooth muscle cells, the formation of elastic lamina and an adventitial layer of connective tissue. The layer of vascular smooth muscle cells provides vasomotor control and structural strength and integrity. Arterioles are to be contrasted with capillary networks. Capillary beds are required for nutrient and gas exchange in the tissue. Because of their small diameter, however, capillaries cannot always satisfy the demand by the tissue for high volume blood flow. This demand can frequently only be met by larger-diameter arteries.

Strategies for stimulation of angiogenesis and arteriogenesis have focussed on delivery of the necessary biological peptides to a localized region of ischemic tissue. For example, delivery of basic fibroblast growth factor (bFGF) to compromised arteries produce changes suggesting improved blood flow (Laham R. J. et al., Clin. Cardiol. 22(Supp. 1): 16-19, (1999). Administration of vascular endothelial growth factor (VEGF) improve myocardial blood flow and has been proposed for use in promoting vascular tissue repair (EP-A-506 477). Other biological factors proposed for use in promoting angiogenesis and/or arteriogenesis include placenta growth factor (WO 01/57181; WO 01/56593) and colony stimulating factor (WO 99/17798). Also described in the art is a method which involves loading monocytes with a therapeutic agent to stimulate arteriogenesis (WO 00/60054). Still other approaches to bring about angiogenesis in patients with ischemic limbs (Isner, J. M. et al., Lancet, 348: 370-374, (1996)) and myocardial ischemia have employed arterial gene transfer of ph VEGF (Losorda, D. W. et al., Circulation, 98(25): 2800-2804, (1996)).

Strategies to replace necrotic, scarred tissue zones of the heart and skeletal muscle by transplanting cardiomyocytes or skeletal myoblasts (Leor, J. et al., Circulation, 94(Supp. 11): 331-336, (1996); Murry et al., J. Clin. Invest., 98: 2512-2523, (1996); Taylor et al., Nat. Med., 4: 929-933, (1998); Tomita et al., Circulation, 100(Supp. 11): 247-256, (1999); Menusche et al., Lancet, 357: 279, (2000)) have shown some success in the survival of some of the grafted cells. However, they have failed to reconstitute healthy myocardium or skeletal muscle and coronary or peripheral arteries which are integrated functionally with the viable healthy tissue.

These approaches reflect the early attempts at a therapeutic approach to ischemia through cell transplantation and promotion of angiogenesis and arteriogenesis. Despite some encouraging findings, none of these approaches have restored blood flow or restored the cellular structural integrity to the ischemic regions to a significant degree to clinically improve the physiological performance of the ischemic regions. There remains therefore an urgent need to bring about angiogenesis, arteriogenesis, and parenchymal cell restoration to a significant degree to be clinically useful.

Compositions and methods for promoting parenchymal tissue restoration and blood circulation in tissue at risk of ischemia, or in post-ischemic tissue, or in hypoxic tissue, are provided herein. The compositions described are effective to stimulate angiogenesis, arteriogenesis, and cardiac and skeletal muscle fiber regeneration as a therapeutic modality.

1. Therapeutic Agents for Promoting Parenchymal Cell Regeneration, Angiogenesis and/or Arteriogenesis

As noted above, the composition includes one or more therapeutic agents effective to attract stem cells and/or progenitor cells, and, optionally, accessory cells, to stimulate the cells into action, and to influence survival of the cells. For a composition designed for treatment of ischemic tissue the therapeutic agents more specifically are selected to (i) attract circulating blood cells including bone marrow cells, stem cells, and progenitor cells, and monocytes (functioning as accessory cells) to a site of ischemia or to the tissue at risk of ischemia, (ii) stimulate a variety of phenomena in these cells, which includes mitogenesis, motility, attachment, differentiation, release of biological agents that promote angiogenesis, arteriogenesis, and the differentiation of bone marrow and stem cells into cardiac myocytes and skeletal myofibers, and (iii) influence the survival of monocyte-derived macrophages, differentiated myocytes and myofibers, precursor cells of myocytes, myoblasts, smooth muscle cells, and endothelial cells in the tissue. As is known, monocytes descend from bone marrow derived myeloid progenitors and belong to the mononuclear phagocyte system. These cells, a category of white blood cells, circulate in the blood and are capable of migrating from the bloodstream through the endothelial layer and into the tissue space where they mature into macrophages. There, macrophages phagocytose and digest invading microorganisms and foreign bodies, as well as damaged and senescent cells. These cells also aid in the remodeling of arterioles to arteries by secreting proteolytic enzymes which open channels in the extra cellular matrix to facilitate the proliferation and migration of smooth muscle cells and the accommodation of the remodeled arteries. As used herein, “monocyte” refers to monocytes as well as other cells that display the same or similar biological actions of monocytes.

Cardiac myocytes and skeletal myoblasts descend from bone marrow stem cells and stem cells which remain in muscle. They differentiate from muscle stem cells which resemble myoblasts and are called satellite cells. Satellite cells enter mitosis and several fuse together to form differentiated muscle fibers. These cells can be attracted to the tissue region of interest by the presence of the appropriate chemoatractant, to begin the process of the regeneration of healthy muscle tissue in ischemic regions and regions at risk for ischemia.

Thus, one class of therapeutic agent included in the composition of the invention for use in ischemic tissue is a biological agent effective to attract circulating blood monocytes, bone marrow stem cells, myogenic progenitors derived from bone marrow, precursor endothelial and smooth muscle cells to the tissue at risk. Such an agent can be a signaling molecule, such as a chemoattractant, effective to attract circulating monocytes, bone marrow stem cells, bone marrow and muscle derived myogenic progenitor cells, precursor endothelial and smooth muscle cells to the tissue region and migrate from circulation to the tissue region. Chemoattractants suitable for use include, but are not limited to, chemokines, such as macrophage chemoattractant proteins (MCP-1, MCP-2, MCP-3, MCP-4, MCP-5), RANTES (regulated upon activation, normal T-cell expressed and secreted cytokine), Fraktalkines, macrophage inflammatory protein (MIP)-1-alpha and MIP-1 -beta; N-farnesyl peptides, complement activation product C5a, leukotriene B4, platelet activating factor (PAF), stem cell factor (SCF), hepatocyte growth factor (HGF), basic fibroblast growth factor (bFGF), platelet-derived growth factors AB and BB (PDGF-AB, BB), leukemia inhibitory factor (LIF), and functionally equivalent fragments of these chemoattractants. Also contemplated for use as attractants for monocytes, bone marrow stem cells, bone marrow and muscle derived myogenic progenitor cells, precursor endothelial and smooth muscle cells are transforming growth factor beta (TGF-beta), interleukins, and colony stimulating factors, such as granulocyte macrophage colony-stimulating factor (GM-CSF), granulocyte colony-stimulating factor (G-CSF), colony stimulating factor-1 (CSF-1) and macrophage colony-stimulating factor (M-CSF), and functionally equivalent fragments thereof.

It will be appreciated that the chemoattractant may be suitable to attract other cells involved in the processes of angiogenesis and/or arteriogenesis, or the chemoattractant can be specifically selected to attract such other cells, in addition to monocytes, bone marrow stem cells, bone marrow and muscle derived myogenic progenitor cells, precursor endothelial and smooth muscle cells. Circulating cells also involved in angiogenesis, arteriogenesis, and muscle cell development include other leukocytes, platelets, bone marrow-derived leukocyte precursors, bone marrow-derived stem cells, and vascular endothelial cell precursors. Vascular smooth muscle cells (VSMC) differentiate from a variety of precursors including bone marrow stem cells, macrophages and local tissue mesenchymal cells. Contemplated for use as stimulants for smooth muscle cells and endothelial cells are GM-CSF, G-CSF, M-CSF, SCF, and Platelet Derived Growth Factor (PDGF-BB).

Table 1 summarizes various growth factors that have a stimulatory effect of cardiac and skeletal muscle cells and their precursors. These factors are suitable as chemoattractants, or as an agent to further proliferation and differentiation of certain cells.

TABLE 1

| Factor | Full Factor Name | Activity |
|---|---|---|
| bFGF | basic fibroblast growth factor | induces proliferation and migration of myoblasts; |
| | | stimulates myogenesis with mesenchymal cell |
| | | recruitment; facilitates differentiation of myoblasts |
| HGF | Hepatocyte growth factor | induces proliferation and migration of myoblasts; |
| | | increases myoblast proliferation and increases the |
| | | number of cells in myotubes; enhances the ability of |
| | | myogenic cells to migrate |
| IGF-1 | Insulin-like growth factor 1 | promotes cell survival of skeletal myoblasts |
| IGF | Insulin-like growth factor | induces cardiac myocyte progenitor mitogenesis |
| | | and differentiation into myotubes; promotes survival |
| | | of skeletal myoblasts and differentiation into |
| | | myotubes |
| EPO | Erythropoietin | suppresses skeletal myoblast apoptosis; stimulates |
| | | proliferation of myoblasts to expand the progenitor |
| | | population during differentiation; has a role in |
| | | muscle development and repair |
| LIF | Leukemia inhibitory factor | potent neuromuscular activity; enhances survival of |
| | | motor and sensory nerves; increases muscle |
| | | regeneration; promotes mitogenesis and |
| | | differentiation of skeletal myoblasts |
| NGF | nerve growth factor | potent stimulator of proliferation and fusion of |
| | | myoblasts forming myotubes |

Another therapeutic agent in the composition is an agent capable of stimulating release of biological agents that promote angiogenesis and/or arteriogenesis, referred to herein as a “stimulation agent”. It will be appreciated that this stimulation agent can be the same agent as the one for use in attracting monocyte, stem cells, and myogenic progenitor cells to the tissue of interest, or a different agent. The stimulation agent is effective to stimulate monocytes, stem cells, or other angiogenesis and arteriogenesis cells in or adjacent to the tissue at risk to release one or more biological compounds that participate in the process of angiogenesis, arteriogenesis, and muscle cell regeneration. Cells other than monocytes involved in the angiogenesis, arteriogenesis, and muscle cell regeneration processes include endothelial cells, mast cells, lymphocytes, granulocytes, leukocytes, platelets, bone marrow-derived leukocyte precursors, bone marrow-derived stem cells, vascular endothelial cells, vascular smooth muscle cells and their precursors.

It will be further appreciated that the stimulation agent may, as well, effect arteriogenesis, angiogenesis, and muscle parenchymal regeneration by direct action on monocytes, other accessory cells, stem cells, and myocyte progenitor cells by direct action on these cells. As illustrated in Table 1, many of the agents that are known to stimulate proliferation and differentiation of cardiac and skeletal myocyte precursor cells to form regenerated cardiac and skeletal myofibers accomplish their action directly upon their targets. Regeneration of muscle parenchyma may occur without stimulation of production and secretion of intermediary biological factors. Similarly, many agents that stimulate angiogenesis and arteriogenesis achieve their effects by direct action upon vascular endothelial cells and their progenitors, and vascular smooth muscle cells and their precursors

The stimulation agent can be a biological or non-biological compound that stimulates the production of angiogenic and arteriogenic biologically active molecules from macrophages and other angiogenic and arteriogenic cells or that stimulates the differentiation of stem cells and the proliferation and survival of muscle cells and the fusion of myoblasts. Exemplary biological agents include, but are not limited to, chemokines, such as macrophage chemoattractant proteins (MCP-1, MCP-2, MCP-3, MCP-4, MCP-5), tumor necrosis factor (TNF-α, TNF-β), RANTES (regulated upon activation, normal T-cell expressed and secreted cytokine), Fraktalkines, macrophage inflammatory protein (MIP)-1-alpha and MIP-1-beta, and functionally equivalent fragments of these agents. Also contemplated for use as stimulation agents are interleukins, colony stimulating factors, such as granulocyte macrophage colony-stimulating factor (GM-CSF), granulocyte colony-stimulating factor (G-CSF), colony stimulating factor-1 (CSF-1) and macrophage colony-stimulating factor (M-CSF), platelet derived growth factors (PDGF-AB and PDGF-BB), basic fibroblast growth factor (b-FGF), insulin-like growth factor (IGF-1), nerve growth factor (NFG), and functionally equivalent fragments of any of these agents. Lipopolysaccharide (LPS), a derivative of some bacterial cell walls may also be suitable for use as an effective stimulant, as would be LPS-like molecules that have the macrophage stimulant effect of LPS but lack the toxic region of the molecule. Also contemplated for use as stimulation agents are angiopoietin-1 and -2 (Ang-1 and Ang-2), hepatocyte growth factor (HGF), leukotriene B4, complement activation products C3a and C5a, leukemia inhibitory factor (LIF), erythropoeitin (Epo), 5-azacytidine, and transforming growth factor (TGF-beta).

In addition to an agent(s) effective to attract circulating monocytes, bone marrow stem cells, bone marrow and muscle derived myogenic progenitor cells, precursor endothelial and smooth muscle cells to the tissue of concern and to stimulate angiogenesis, arteriogenesis, and muscle parencyma regeneration by direct action or through the induction of secretion of biologically active agents, the composition can further include an agent effective to influence the survival of cells in the tissue of concern that are participating in the regeneration of blood vasculature and muscle parenchyma. For example, extending the survival of macrophages that derive from the monocytes attracted to the tissue region and extending the survival of other angiogenic and arteriogenic cells in the tissue region will promote and extend the release of desired agents involved in angiogenesis and/or arteriogenesis. The composition can also include an agent effective to increase the number of circulating stem cells which participate in angiogenesis and arteriogenesis and which can differentiate into cardiac and skeletal muscle cells. Biological agents capable of influencing the survival of circulating blood monocytes and of monocyte-derived macrophages resident in tissue, other cells contributing to angiogenesis and arteriogenesis and which increase the number of stem cells include GM-CSF, G-CSF, CSF-1, M-CSF, IGF-1, Ang-1, and functionally equivalent agents and fragments of these.

The composition can also include one or more biological agents involved in the process of angiogenesis and arteriogenesis. These agents are typically released from the macrophages and other cells in the ischemic tissue, but could also be provided as part of the therapeutic composition. Factors involved in angiogenesis and/or arteriogenesis include fibroblast growth factor (FGF, FGF-1, FGF-2), TGF-alpha, insulin-like growth factor-1 (IGF-1), angiopoietin-1 (Ang-1), angiopoietin-2 (Ang-2), vascular endothelial growth factor (VEGF), functionally equivalent fragments thereof, constructs of VEGF such as VEGF-2, VEGF165, and VEGF121, platelet derived growth factor-A (PDGF-A) and/or PDGF-B and/or PDGF-BB, placental-derived growth factor (PIGF), and other endothelial mitogenic growth factors, or functionally equivalent fragments thereof.

The composition can also include an agent to assist in binding of circulating monocytes, macrophages, or other cells, to the tissue region. The agent can also function to bind or closely associate the macrophages with the drug reservoir, to bring the macrophage in proximity with the agent(s) being released from the reservoir. In one embodiment, the composition further includes a cellular adhesion molecule, such as ICAM, VCAM, PECAM; VE-cadherin; fibronectin fragments, such as RGD and REDV; synthetic adhesion peptides, such as VAPG and KQAGDV; extracellular matrix molecules and heterogeneous mixtures of collagen, fibronectin, heparin, dextrans, hylans, laminin, and processed extracellular matrix (ECM) from animal origin. Such an agent is associated with the drug reservoir, described below, in various ways, including entrapping or enclosing the agent within the reservoir, coating the surface or attaching the agent to the external surface of the reservoir.

2. Cardiac Muscle Regeneration

As can be appreciated from the above discussion, treatment of cardiac tissue at risk of ischemia or post-ischemia is achieved with the composition described herein. In addition to ischemia due to infarct or stroke, cardiac tissue is susceptible to damage from other conditions, such as cardiomyopthathies of inflammatory, toxic, metabolic, and congenital etiologies, and congestive heart failure. The exemplary compositions described above suitable for ischemic cardiac tissue may also find use in treating cardiac tissue damaged by a condition other than ischemia. Additionally, compositions for treatment of damaged myocardium can be designed based on the following discussion.

As noted above, the composition of the invention requires biological factors capable of three functions, chemoattraction of stem cells, progenitor cells, and accessory cells, stimulation of such cells, and enhancing the survival of these cells and the differentiated cells of regenerated myocardium Compositions tailored for treatment of cardiac tissue will involve attraction of progenitor cells including cardiomyoblasts (cardiomyocytes), bone marrow stem cells, hematopoietic stem cells, marrow stromal cells, resident cardiomyocyte stem cells. Endothelial cells, vascular smooth muscle cells, and their precursors will also be involved in the tissue repair process. Accordingly, the composition will include at least one factor that serves as a chemoattractant for one or more of these progenitor cells. A number of exemplary chemoattractants are given above.

The composition will additionally include at least one factor that acts to stimulate the progenitor cells into action, e.g., to differentiate and/or proliferate. Suitable stimulation factors include but are not limited to stem cell factor, insulin-like growth factor-1 and -2 (IGF-1, IGF-2), transforming growth factor-α, -β (TGF-α, TGF-β), heparin-binding epidermal growth factor-like growth factor (HB-EGF), and 5-azacytidine.

The composition will also include at least one factor that acts to facilitate survival of the progenitor cells and their differentiated progeny, and exemplary stimulation agents include, but are not limited to, include GM-CSF, G-CSF, CSF-1, M-CSF, and IGF-1.

The composition will also include cell adhesion molecule ligands that bind to cell surface integrins on stem cells (in particular VLA4) and progenitor cells. An exemplary ligand is vascular cell adhesion molecule 1 (VCAM-1). Adhesion by such a ligand ensures localization of stem cells to composition depositing sites in damaged tissue.

3. Skeletal Muscle Regeneration

In another embodiment, a composition designed for repair of skeletal muscle by production of myofibers is contemplated. Compositions are contemplated which would be appropriate for regeneration of skeletal muscle damaged, for example, by thermal injury, Duchenne's muscular dystrophy, physical crush injury, obstructive peripheral disease, and other insults.

Progenitor cells involved in the repair process include bone marrow stem cells, hematopoietic stem cells, marrow stromal cells, skeletal muscle satellite cells, myoblasts, peripheral blood cells, and muscle precursor cells. Attraction of these cells to a site of skeletal muscle damage is achieved by introducing a composition containing a chemoattractant. Exemplary attractants include growth factors, such as basic fibroblast growth factor (bFGF), hepatocyte growth factor (HGF), transforming growth factor-3 (TGF-β), platelet-derived growth factor (PDGF), platelet-derived growth factor-AB (PDGF-AB), platelet-derived growth factor-BB (PDGF-BB), interleukin-8 (IL-8), tumor necrosis factor-α (TNF-α), leukotrine B4, fibronectin (Fn) fragments, complement activation products C3a and C5a, leukemia inhibitory factor (LIF), and regulated on activation normal T-cell expressed and secreted cytokine (RANTES).

The composition will also include at least one factor to stimulate the attracted cells into action. Suitable factors include, but are not limited to, insulin-like growth factor-I (IGF-I), insulin-like growth factor-II (IGF-II), basic fibroblast growth factor (bFGF), nerve growth factor (NGF), hepatocyte growth factor (HGF), platelet-derived growth factor (PDGF), platelet-derived growth factor-BB (PDGF-BB), transforming growth factor β (TGF-β), erythropoeitin (EPo), human leukemia inhibitory factor (hLIF), and 5-azacytidine.

The composition will also include one or more factors that prolong the survival of the progenitor cells and differentiated cells of regenerated (ion) parenchyma. Factors that prolong survival of cells in skeletal muscle tissue include, insulin-like growth factor-I (IGF-I), insulin-like growth factor-II (IGF-II), platelet-derived growth factor-BB (PDGF-BB), and erythropoeitin (EPO).

The composition will also include cell adhesion molecule ligands that bind to cell surface integrins on stem cells and progenitor cells. Exemplary ligands include vascular cell adhesion molecule 1 (VCAM-1), Matrigel, and fibronectin fragments (Fn). Adhesion by such a ligand ensures localization of stem cells to composition depositing sites in damaged tissue. Matrigel is also known to stabilize and preserve the activity of many of the cytokines contemplated above as stimulant factors, and act to present these cytokine ligands to receptors on stem cell and progenitor cell surfaces to produce optimum stimulation.

4. Tissue Regeneration in the Liver

The liver is the target of attack for a wide range of diseases. These diseases include infectious, autoimmune, as well as non-infectious processes such as chemical toxicity. Examples of infectious diseases include: (i) viral hepatitis, e.g., hepatitis A, B, C, D, E, and G and (ii) parasitic hepatitis, e.g., Schistosoma mansoni, Schistosoma hematobium, and Schistosoma japonicum. (Harrison's Principles of Internal Medicine, Fauci et al. eds., 1998, pgs 1660-1725). Examples of noninfectious diseases affecting the liver, include autoimmune diseases, such as, (i) autoimmune hepatitis and (ii) primary biliary cirrhosis. (Harrison's Principles of Internal Medicine, Fauci et al. eds., 1998, pgs 1701-1709). Regardless of whether the attack on the liver is infectious, autoimmune or noninfectious, if left untreated damage to the liver causing scarring or fibrosis will result. The end-stage of fibrosis is cirrhosis. Pathologically, cirrhosis is defined as extensive fibrosis in the liver in association with the formation of regenerative nodules. Cirrhosis is the final common pathway for many, if not all, types of chronic liver damage and is typically progressive. Although the liver in some situations has tremendous capacity to regenerate itself, liver damage can inhibit or abolish this regenerative capacity. Accordingly, the invention contemplates deposition of a composition to effect regeneration of liver tissue.

Stem cells and progenitor cells involved in liver regeneration include hepatocytes, bone marrow cells, liver precursor stem cells, liver adult stem cells, liver epithelial ductal cells, and oval cells. Attraction of these cells to a site of tissue trauma is achieved by introducing a composition containing a chemoattractant. Exemplary attractants include granulocyte colony stimulating factor (G-CSF), interleukin-8 (IL-8), stromal cell derived factor (SDF-1), stem cell factor (SCF), sulfated polysaccharides such as flucoidan and all the chemoattractants noted above for use in cardiac muscle, skeletal muscle, and for attracting stem cells and progenitor cells for promoting angiogenesis and/or arteriogenis.

The composition will also include at least one factor to stimulate the attracted cells into action. Suitable factors include, but are not limited to, insulin-like growth factor-I (IGF-I), insulin-like growth factor-II (IGF-II), epidermal growth factor (EGF), basic fibroblast growth factor (bFGF), hepatocyte growth factor (HGF), keratinocyte growth factor (KGF), activin-B, interleukin-6 (IL-6), tumor necrosis factor (TNF), lipo-polysaccharide (LPS), transforming growth factors (TGF-α), transcription factors include nuclear factor for the kappa chain of B cells (NFKB), STAT3, AP-1, C/EBPs, and hepatic stimulatory substance (HSS).

The composition will also include one or more factors that prolong the survival of the progenitor cells and the differentiated cells of the regenerated liver parenchyma. Factors that prolong survival of cells in liver tissue include, interleukin-6 (IL-6) and tumor necrosis factor (TNF).

5. Tissue Regeneration in the Pancreas

The human pancreas is a gland comprised of both exocrine and endocrine tissues, with the exocrine tissues involved in secretion of digestive enzymes and the endocrine tissues involved in production of insulin, glucagon, somatostatin, and pancreatic polypeptide. Insulin and glucagon act in concert to regulate the production and metabolism of glucose. The endocrine pancreas comprises the pancreatic islets of Langerhans which are aggregations of polypeptide hormone producing cells scattered widely throughout the acinar tissue and which are most numerous in the tail portion of the pancreas. Typically, total islet tissue constitutes only about 1 or 2 percent of the pancreatic mass.

Islet tissue contains at least three functionally different types of cells: A (or “alpha”) cells which can make glucagon, B (or “beta”) cells which make insulin, and D (or “delta”) cells which can make a third islet hormone, somatostatin, and PP cells, which secrete pancreatic polypeptide. The B cells are the most abundant of the three types of islet cells. Insulin promotes the uptake of glucose by cells, especially muscle cells, and prevents an excessive breakdown of glycogen stored in liver and muscle.

Diabetes affects 4 to 5% of the population worldwide and is the most common metabolic disorder. The number of individuals diagnosed with diabetes is rapidly increasing, especially in the developed countries and the disorder frequently leads to secondary complications such as retinopathy, nephropathy, neuropathy and cardiovascular disease. Type II (non-insulin-dependent) diabetes mellitus is the most common form of diabetes, more than 90% of diagnosed cases, and results from insulin resistance, pancreatic beta-cell dysfunction, or a combination of both. The beta-cell dysfunction seems to result in part from an inability of the beta cells to produce and secrete sufficient amounts of active insulin in response to an increased demand for insulin. Type I (insulin-dependent) diabetes mellitus is caused by an autoimmune destruction of the insulin producing beta cells, resulting in insulin deficiency. The existing therapies for both types of diabetes may require daily administrations of insulin. These therapies are unsatisfactory since they do not offer a cure and are mostly insufficient for preventing the secondary complications associated with diabetes.

Strategies to replace beta cells of the pancreas that produce insulin have focused on allogenic pancreatic islet cell and stem cell transplantation. All transplantation from donors that are different from the recipient require chronic administration of immunosuppresant drugs to prevent graft tissue rejection of graft host disease, screening of the graft donor for communicable bacterial and viral infections and genetic screening for a predisposition to other debilitating diseases.

Damage to pancreatic tissue can result from diabetes. Accordingly, the invention contemplates deposition of a composition to effect regeneration or repair of pancreatic tissue.

Cells involved in regeneration of damaged pancreatic tissue include beta cells, islet stem cells, islet precursor cells, and pancreatic ductal stem cells. Attraction of these cells to a site of tissue damage is achieved by introducing a composition containing a chemoattractant. Exemplary attractants include vascular endothelial growth factor (VEGF), granulocyte colony stimulating factor (G-CSF), interleukin-8 (IL-8), stromal cell-derived factor-1 (SDF-1), stem cell factor (SCF), flucoidan, and extracellular matrix (ECM).

The composition will also include at least one factor to stimulate the attracted cells into action. Suitable factors include, but are not limited to, nerve growth factor (NGF), basic fibroblast growth factor (bFGF), vascular endothelial growth factor (VEGF), insulin-like growth factor-I (IGF-I), insulin-like growth factor-II (IGF-II), hepatocyte growth factor (HGF), platelet-derived growth factor (PDGF), transforming growth factor-β (TGF-β), epidermal growth factor (EGF), keratinocyte growth factor (KGF), activin-A, extendin-4, growth hormone, prolactin, placental lactogen, nicotinamide, betacellulin (BTC), leukemia inhibitory factor (LIF), macrophage migration inhibitory factor (MIF), islet factor-1 (Isl-1), pancreatic duodenal homeobox-1 (Pdx-1), pancreatic duodenal homeobox-4 (Pdx-4), pancreatic duodenal homeobox-6 (Pdx-6), cdx-2, Nkx-6, and hepatocyte nuclear factor (HNF-1α).

The composition will also include one or more factors that prolong the survival of the stem cells, progenitor cells, and differentiated cells of regenerated tissue. An exemplary factor that prolongs survival of cells in pancreatic tissue is insulin-like growth factor-I (IGF I), nerve growth factor (NGF) and lnterleukin-6 (IL-6).

The composition can also include ligands to which the surfaces of stem cells and progenitor cells can bind. These ligands are contemplated to increase efficiency of regeneration by increasing the number of cells in close proximity to the stimulating factors that are also part of the composition. It is known that binding of stem cells and progenitor cells to cell adhesion moieties of the extracellular matrix promotes mitogenesis and differentiation. Suitable ligands include N, R, E-cadherins, and other cell adhesion molecules (CAM), including neural cell adhesion molecules (NCAM).

6. Tissue Regeneration in the Central Nervous System

Damage to brain and spinal cord tissue can also result from a variety of situations and conditions, which include infections (such as the various bacterial and viral meningoencephalitides), vascular disorders (such as hemorrhagic and ischemic stroke), degenerative disorders (such as multiple sclerosis, Parkinson's disease Alzheimer's disease) and physical trauma, including concussion of the brain, laceration of the brain, and pressure and crush lesions to the spinal cord.

Accordingly, in one embodiment, the invention contemplates use of the composition for regeneration of brain and spinal cord tissue post-damage or at risk of damage. In particular, the composition will include a first agent effective to attract essential cells, including but not limited to microglia, oligodendroglia, neural adult stem cells, neurons, bone marrow (BM) cells, accessory cells (AC), smooth muscle cells (SMC), marrow stromal cells (mSC), hematopoietic bone marrow stem cells, (hSC), and astroctyes. Some of these cells can migrate across the blood brain barrier (BBB) and/or are present in the brain tissue. Attraction of accessory cells is known to be essential In the response of the brain to damage. It is known that marrow stromal cells (mSC), marrow hematopoietic stem cells (hSC), other bone marrow (BM) cells, microglia, astroglia, and monocyte-macrophages enter the damaged tissue regions and act as accessory cells (AC), by producing a variety of cytokines and other biological factors which directly induce mitogenesis of stem cells and progenitor cells, and differentiation of progenitors to functioning glia and neurons. Agents effective to attract one or more of these above cells include hepatocyte growth factor (HGF), macrophage chemoattractant protein1-1 (MCP-1), stromal cell-derived factor-1α (SDF-1α), stromal cell-derived factor-1β (SDF-1β), basic fibroblast growth factor (bFGF), epidermal growth factor (EGF), interleukin-1 (IL-1), and platelet-derived growth factor-AB (PDGF-AB).

A second agent is included in the composition to stimulate a variety of phenomena in the attracted cells, which include: (I) proliferation of stem cells and progenitor cells, (ii) differentiation to functional parenchymal cells, and (iii) production of a variety of cytokines and other biological agents which stimulate proliferation, differentiation, and modulate and coordinate differentiation amongst regenerating glia and neurons. Exemplary stimulatory agents include, but are not limited to neurotrophin 3 (NT3), brain-derived neurotrophic factor (BDNF), nerve growth factor (NGF), epidermal growth factor (EGF), basic fibroblast growth factor (bFGF), NEP1-40 inhibitor of Nogo protein, neurotrophin 4 (NT4), β-mercaptoethanol (β-ME), human leukemia inhibitory factor (hLIF), retinoic acid (RA), interleukin-1 (IL-1), interleukin-6 (IL-6), platelet-derived growth factor-AB (PDGF-AB), transforming growth factor-α (TGF-α), stem cell factor (SCF), vascular endothelial growth factor (VEGF), insulin, forskolin, valproic acid, heparin, heparan, glycosylated cytostatin-C, and phorbol myristate acetate (TPA).

It is contemplated that the composition will, additionally, include agents which act directly on the tissue extracellular matrix and the blood-brain barrier to allow complete development of regenerated nervous system parenchyma. These agents may act to implement neuronal plasticity, in which parenchymal regeneration is accomplished by re-connection of neuronal networks in configurations different from those in the damaged tissue. Exemplary agents to accomplish these components of regeneration include neurotrophin 3 (NT3), chondroitinase ABC (chABC), NEP1-40 inhibitor of Nogo protein binding, and vascular endothelial growth factor A (VEGF-A).

The composition will also include one or more factors that prolong the survival of the stem cells, progenitor cells, and/or differentiated cells. Factors that prolong survival of cells in brain tissue include, brain-derived neurotrophic factor (BDNF), nerve growth factor (NGF), basic fibroblast growth factor (bFGF), epidermal growth factor (EGF), platelet-derived growth factor-AB (PDGF-AB), glycosylated cytostatin-C, β-mercaptoethanol (β-ME), butylated hydroxyanisole (BHA), dimethyl sulfoxide (DMSO), hepatocyte growth factor (HGF), nerve growth factor, neurotrophin 4 (NT4), butylated hydroxytoluene (BHT), and human leukemia inhibitory factor (hLIF).

The composition can also include ligands to which the surfaces of stem cells, progenitor cells, and other cells can bind. These ligands are contemplated to increase efficiency of regeneration by increasing the number of cells in close proximity to the stimulating factors that are also part of the composition. It is known that binding of stem cells and progenitor cells to cell adhesion moieties of the extracellular matrix promotes mitogenesis and differentiation. Suitable ligands include laminin and vascular cell adhesion molecule 1 (VCAM-1).

7. Tissue Regeneration in the Kidney

In another embodiment, the invention includes tissue regeneration in the kidney. A variety of diseases can cause damage to kidney parenchyma, such as atheroembolic disease, renal vein thrombosis, renal artery embolism, thrombosis, diabetic nephropathy, glomerulonephritides of various etiology, toxic nephrosis, and pyelonephritis. As a result of the damage, renal failure, whether arising from an acute or chronic decline in renal function, is a grave condition that can result in substantial or complete failure of the filtration, reabsorption, endocrine, and homeostatic functions of the kidney.

Accordingly, deposition of a composition containing therapeutic agents effective to attract cells capable of differentiation or proliferating into cells that could supply some or all of the functions provided by the kidney and that are capable of producing functional renal cells or regenerating a functioning kidney, in whole or in part is contemplated. Stem cells in the kidney are capable of contributing to the formation of metanephric tubule cells. It is known that bone marrow stromal cells (mSC) can travel through the peripheral blood from the marrow to sites of damage resulting from glomerulonephritis and contribute to the mesenchymal cell structure that is integral to the function of the nephron (the fundamental functional unit of the kidney) in regenerating kidney. Hematopoietic stem cells can differentiate into glomerular mesangial cells. Differentation and proliferation are facilitated by agents such as interleukin-11 (IL-11) and steel factor. Deposition of a composition containing a chemoattractant to draw stem cells or precursor cells capable of differentiation into cells forming kidney tissue, along with agents to facilitate the differentiation and proliferation of the cells, and an agent to enhance survival of stem cells, progenitor cells, and differentiated cells would result in repair of the damaged kidney tissue, by, for example, regeneration of various cells of the kidney such as mesangial cells, podocytes, epithelial cells, smooth muscle cells, and endothelial cells.

8. Tissue Regeneration Composition and Stem Cell Implantation

Stem cell transplantation as a therapy for treatment of various disorders, such a leukemias and multiple myeloma, is widely investigated. In another embodiment of the present invention, deposition of the composition containing chemoattractants for stem cells and progenitor cells, factors to stimulate the cells to differentiate or proliferate, and factors to enhance survival of the cells, in conjunction with stem cell transplantation is contemplated. In this embodiment, factors are selected according to the disorder and/or tissue to be treated. Selection of suitable factors can be determined based on the guidance provided in the above description.

B. Compositions for Attraction of Immune Cells to Tumor Site

The invention provides, in another aspect, compositions for promoting cytotoxic attack upon tumor cells by tumor infiltrating lymphocytes (TIL) within a primary tumor and/or within metastasis. The composition, upon deposition at the site of the primary tumor or a metastasis, promotes tumor regression and, ultimately, tumor destruction. The composition is effective to stimulate a cytotoxic, systemic immunlogical response by an otherwise naive host, the stimulated response being directed at tumor antigens in either a primary tumor or a matastasis. The compostion includes one or more drug reservoirs suitable for deposition at tumor site, the reservoirs containing (i) chemoattractants for T lymphocytes, including cytotoxic and helper T cells, dendritic and natural killer cells, their progenitor cells and accessory cells, such as eosinophils and tumoricidal macrophages; (ii) agents to stimulate the cells to become activated and to differentiate and proliferate; and (iii) and agents effective to enhance survival of the cells and provide immunity.

More specifically, the composition includes one or more therapeutic agents effective to (i) attract lymphocytes (including all subsets of naive T lymphocytes and natural killer lymphocytes), macrophages, polymorphonuclear leucocytes, and antigen-presenting cells (including dendritic cells) to infiltrate the tumor mass and gain direct access to viable and necrotic tumor cells; (ii) stimulate an inflammatory response within the tumor mass which will be the setting for presentation of tumor antigens to lymphocytes which will culminate in proliferation of populations of cytotoxic and natural killer (NK) lymphocytes generally available within the body for tumor attack; and (iii) promote survival of the attracted cells, to allow trafficking of cytotoxic and natural killer lymphocytes in an immune host into the tumor mass for direct access and killing of tumor cells.

In one embodiment, the one or more agents effective to attract one or more cells to the tumor site include GM-CSF, interleukin-12 (IL-12), secondary lymphoid tissue chemokine (SLC), monocyte chemoattractant protein (MCP), monokine induced by IFN gamma (MIG) tumor necrosis factor (TNF), macrophage inflammatory protein protein (MIP), stromal cell-derived factor-1α (SDF-1α), stromal cell-derived factor-1β (SDF-1β), RANTES and interleukin-1 (IL-1). Preferred chemoattractive agents are chemoattractive agents including IL-8, MIG, IL-12, MCP-1, -2, -3, -4, -5, MIP-1 alpha, MIP-1 beta, MIP-1 gamma, and RANTES.

The second agent is able to to stimulate a variety of phenomena in the attracted cells, which include: (i) proliferation of T lymphocytes, including cytotoxic and helper T cells, dendritic and natural killer cells, lymphokine-activated killer cells, their progenitor cells and accessory cells such as eosinophils and tumoricidal macrophages and their progenitor cells; (ii) differentiation to functional and activated immune and accessory cells; and (iii) production of a variety of cytokines and other biological agents which stimulate proliferation, differentiation, and modulate and coordinate differentiation amongst immune system progenitor cells, promote the survival of the immune and accessory cells and enhances the cytotoxic killing potential of the immune cells. Exemplary stimulatory agents include, but are not limited to, interleukin-1 (IL-1 ),interleukin-2 (IL-2), interleukin-6 (IL-6), interleukin-7 (IL-7), interluekin-12 (IL-12), and GM-CSF.

The third agent in the composition for cancer immunotherapy is an agent capable of promoting survival of the attracted and local cells, to enhance movement of cytotoxic and natural killer lymphocytes into the tumor mass. Suitable agents for this feature include MIG, platelet factor 4, MCP-1, -2, -3, and MIP-1 gamma.

C. Drug Reservoir

The one or more agents described above are deposited at a tissue region of interest in the form of reservoirs containing the desired agents. The reservoirs contain and release the therapeutic agents to promote the attraction and stimulation of stem cells, progenitor cells, and, optionally, accessory cells, and the survival of accessory cells, progenitor cells, and fully differentiated tissue parenchymal cells. The reservoirs can be designed and synthesized to release in a kinetically desirable mode the associated agents, as will be described.

A first exemplary drug reservoir is a macroporous reservoir comprised of a biologically and chemically inert particle having interconnected pores. The pores are open to the particle surface for communication between the exterior of the particle and the internal pore spaces. Exemplary particles for formation of such macroporous reservoirs are described, for example, in U.S. Pat. No. 5,135,740, incorporated by reference herein.

The particles are typically formed by suspension polymerization in a liquid-liquid system. In general, a solution containing monomers and a polymerization catalyst is formed that is immiscible with water. An inert solvent miscible with the solution but immiscible with water is included in the solution. The solution is then suspended in an aqueous solution, which generally contains additives such as surfactants and dispersants to promote the suspension or emulsion. Once the suspension is established with discrete droplets of the desired size, polymerization is effected, typically by activating the reactants by either increased temperature or irradiation. Once polymerization is complete, the resulting solid particles are recovered from the suspension. The particles are solid, spherical, porous structures, the polymer having formed around the inert liquid, thereby forming the pore network. The inert solvent, which served as a porogen, or pore-forming agent, occupies the pores of the particles. The porogen is subsequently removed and the internal pores are now available to be filled with a therapeutic agent, as described below.

The macroporous particles can also be prepared by solvent evaporation, from either a biodegradable or a non-degradable polymer. For the solvent-evaporation process, the desired polymer is dissolved in an organic solvent and the solution is then poured over a layer of sodium chloride crystals of the desired particle size (Mooney, et al., J. Biomed. Mater. Res. 37: 413-420, (1997)). The solvent is removed, generally by evaporation, and the resulting solid polymer is immersed in water to leach out the sodium chloride, yielding a porous polymer reservoir. Alternatively sodium chloride crystals can be dispersed in the polymer solution by stirring to obtain a uniform dispersion of the sodium chloride crystals. The dispersion is then extruded dropwise into a non-solvent for the polymer while stirring to precipitate the polymer droplets around the sodium chloride crystals. The solid polymer particles are collected by filtration or centrifugation and then immersed in water to leach out the sodium chloride, yielding a porous polymer reservoir. It will be appreciated that alternatives to sodium chloride include any non-toxic water soluble salt or low molecular weight water soluble polymer which can be leached out to produce the desired porosity.

In a study conducted in support of the invention, described in Example 1, polymeric drug reservoirs were prepared. Particles having an average size of 20 μm were formed from butyl methacrylate, ethylene glycol dimethacrylate and polyvinyl alcohol. The particle porosity was 80%. Example 3A details another example, where solid spherical particles of a bioerodible polymer and containing GM-CSF were prepared.

The particles may vary widely in size, from about 0.0001 micron to about 100 microns in diameter, preferably from about 0.001 microns to about 40 microns. The pore dimensions within the particles may also vary widely, with resulting dimensions depending on the chemical characteristics of the polymers used. Typical total pore volumes range from about 0.01 to about 10 cc/g, preferably from about 0.1 to about 6 cc/g; and average pore diameters range from about 0.000001 microns to about 3.0 microns, preferably from about 0.000001 microns to about 1.0 micron.

The particles are loaded with one or more of the therapeutic agents described above by, for example, dissolving the agent in an aqueous solution (buffered to the appropriate pH if necessary), and mixing by stirring the particles and the aqueous solution until all of the liquid is absorbed by the particles to give a free flowing powder. The particles may then be freeze dried to remove the aqueous phase in the particles leaving entrapped in the pores of the particles a lyophilized form of the therapeutic agents. Example 2 describes a study where single biological agents were loaded into porous polymer particles by mixing the particles and a solution of the biological agent. After adsorption of the solution, the particles were lyophylized to remove the aqueous phase, leaving the biological agent entrapped in the pores of the particles. Example 2 also describes preparation of drug reservoirs containing combinations of biological agents.

Example 3 describes another study, where bioerodable polymer spheres containing one or more biological agents were prepared. The bioerodable particles were formed of DL-lactide-co-glycolide and contained a single agent, GM-CSF, or a combination of agents, G-CSF and RANTES. It will be appreicated that the preparation techniques described in Examples 2 and 3 can be used to entrap any desired single agent or combination of agents in porous or solid particles.

An alternative entrapment process for the therapeutic agents in the particles is to first, prior to entrapping the agent in the macroporous particles, add the therapeutic agent(s) to a polymer solution, such as a copolymer of polyethylene oxide-polypropylene oxide-polyethylene oxide (Pluronic®), other water soluble polymers, such as polyvinylpyrilidone, polyvinyl alcohol, or any other non-toxic water-soluble polymer, including biological polymers such as collagen, fibrin, hyaluronate, and the like. The therapeutic agent—polymer aqueous solution is then entrapped in the porous particle as described above. This alternative process offers the benefit of a polymer coating around the lyophilized therapeutic agent which serves to protect the entrapped agents from enzymatic attack when delivered, and to permit a further degree of control of the release of the agent from the porous particle. As can be appreciated, this enhanced control over release of the agent is obtained by selection of the polymer that coats the therapeutic agent. Control over release of the agent is also achieved by selection of the porous particle size, size of the pores, and loading of the agent in the particles. It will further be appreciated that one of several agents entrapped in the porous particles can be coated with a polymer, to slow release of that one agent. It will also be appreciated that the one or more agents in the porous particle can be coated with one or more different coating polymers prior to entrapment in the porous particle, to effect a different release of each agent from the particles.

Another drug reservoir for use in the composition of the invention is a microcapsule and microparticles, where the desired therapeutic agent(s) are contained or dispersed therein. Both microcapsules and microparticles are well known in the pharmaceutical and drug delivery industries (see, for example, Baker, R. W., CONTROLLED RELEASE OF BIOLOGICALLY ACTIVE AGENTS, John Wiley & Sons, NY, 1987; Ranade V. and Hollinger, M., DRUG DELIVERY SYSTEMS, CRC Press, 1996). Microcapsules typically refer to a reservoir of active agent surrounded by a polymer membrane shell. A microparticle typically refers to a monolithic system where the therapeutic agent(s) is dispersed throughout the particle. There are, however, many formulations falling between these two definitions, such as agglomerates of microcapsules, and such formulations would also be suitable for use herein.

Microcapsules and microparticles can be prepared from biodegradable or non-biodegradable polymers. Microcapsules are readily formed by a number of methods, including coacervation, interfacial polymerization, solvent evaporation, and physical encapsulation methods (, Baker, R. W., CONTROLLED RELEASE OF BIOLOGICALLY ACTIVE AGENTS, John Wiley & Sons, NY, 1987). Microparticles are prepared by numerous techniques known in the art, one simple way being to merely grind a polymer film containing dispersed therapeutic agent into a suitable size. Spray drying particulate therapeutic agent from a polymer solution is another approach. Specific procedures for encapsulation of biologically active agents are disclosed in U.S. Pat. No. 4,675,189 and U.S. patent application No. 20010033868, which are incorporated by reference herein.

Another delivery vehicle for use in the present composition is a polymer gel formulation. One particularly suitable polymer for use in such a gel formulation is a polyoxyethylene-polyoxypropylene block copolymer (Pluronic®). These copolymers exhibit reverse thermal gelation behavior, have good drug release characteristics, and have a low toxicity. The copolymers gel as a function of temperature and polymer concentration., where an aqueous solution gels as the solution is warmed. The gel has a low viscosity at room temperature (25° C.), but at an in vivo body temperature (37° C.) the viscosity increases.

To form a composition, desired therapeutic agents are combined with the polymer in a liquid, preferably, an aqueous solution. The solution can be administered via a suitable delivery device, such as the catheter described below, to the target site. Upon delivery to the warmer environment, gelation occurs, localizing and depositing the therapeutic agent at the desired site.

Other suitable polymers for preparation of delivery reservoirs include, but are not limited to collagen (Pieper, J. S. et al., Biomaterials, 21: 1689-1699 (2000)); fibrin (Grassl, E. D. et. al., J. Biomed. Mater. Res., 60: 607-612 (2002)); yaupon gels (Ramamurthi, A. et al., J. Biomed. Mater. Res., 60: 196-205 (2002)); derivatized dextrans (Letourneur, D. et al., J. Biomed. Mater. Res., 60: 94-100 (2002)): heparin alginate (Laham, R. et al., Circulation, 100: 1865-1871 (1999)); alginate (U.S. Pat. Nos. 6,238,705; 6,096,344); and chochleates (U.S. Pat. No. 6,403,056). Drug reservoirs containing the desired therapeutic agents are prepared from these materials by, for example, preparing an aqueous solution of the agent and the polymer and then removing the solvent by freeze-drying to produce a lyophilize form of the biological agent in the reservoir. The lyophilized dry powder can be administered directly, or resuspended in any suitable liquid, such as a low viscosity fluid, a viscous gel or ointment, or a water-in-oil or oil-in-water emulsion. The suspending medium may be aqueous or non-aqueous and is selected based on a number of factors, for example, the time lapse between formulating the reservoirs in the vehicle and time of delivery to the tissue and the desired release kinetics of the biological agent.

Yet another suitable drug reservoir is a liposome. Liposomes are spherical lipid vesicles, ranging in size from 0.01 to 10 microns, and consist of one or more lipid bilayer encapsulating an aqueous space. A variety of amphipathic lipids are used to form the bilayer, such as phospholipids (see, for example, U.S. Pat. No. 5,013,556). The lipid molecules are arranged with their polar head groups toward the water phase and the hydrophobic hydrocarbon tails adjacent to one another in the bilayer, thus forming closed, concentric bimolecular lipid leaflets separating aqueous compartments.

Therapeutic agents can be entrapped within the aqueous interstitial spaces of the liposomes, or within the lipid bilayer, depending on the physiochemical properties of the agent and the composition of the lipid bilayer. Release of the agent(s) from the liposomes can be tailored based on the selection of lipid bilayer components.

In one embodiment of the invention, the drug reservoir containing the therapeutic agents has a biological agent associated with the external surface of the drug reservoir. For example, in a liposomal drug reservoir, the external surface of the liposomes is comprised of the polar head groups of the phospholipids forming the lipid bilayers. The polar head group of a portion of the phospholipids can be derivatized, before or after liposome formation, to include a biological agent, such as a cellular adhesion molecule. The biological agent can be attached directly to the lipid head group or attached though a polymer arm. Both approaches, that is liposomal surface-attached molecules and molecules attached via a spacer arm, have been described in the art (see, for example, U.S. Pat. No. 5,013,556; Zalipsky, S., polyethylene glycol-lipid conjugates in Lasic, D. and Martin, F., Eds. STEALTH LIPOSOMES, CRC Press, 1995; WO 98/16202; WO 94/21281).

It will be appreciated that a biological agent can also be readily attached to the external surface of the drug reservoirs described above. For example, the outer polymer shell of microcapsules or microparticles can be treated to render active certain polymer moieties, for subsequent reaction with and attachment of the desired therapeutic agent to the surface. Alternatively, the therapeutic agent may be coated on the surface of the microcapsules or microparticles. In this way, the therapeutic agent is exposed for immediate activity upon deposition of the composition at the target tissue.

It will also be appreciated that the biological or synthetic polymer from which the drug reservoirs are made may be chosen for its chemical make-up, or it may be derivatized to allow the reservoir and the biological agent to participate in some type of physical attraction holding the therapeutic agent on the surface . In this way, the therapeutic agent is exposed for immediate activity upon deposition of the composition at the target tissue.

As discussed above, the drug reservoirs contain one or more therapeutic agents, such as a chemoattractant, a stimulation agent, and/or a survival-enhancing agent. The release of the one or more agents can be tailored by selection of the drug reservoir and by formulation of the composition. For example, in embodiments where two agents are entrapped in the drug reservoir, the agents can be formulated for simultaneous or sequential release. Release from a microcapsule or a microparticle will be sequential if the entrapped therapeutic agents have significantly different permeabilities to the polymer forming the outer coating of the capsule or to the polymer forming the matrix of the particle. Sequential release from microparticles and microcapsules can also be achieved by, for example, preparing a laminate comprised of different polymers, with the one or more therapeutic agents dispersed in the various laminate layers or entrapped between each laminate layer. Examples of sequential release are disclosed in, for example, U.S. Pat. Nos. 5,472,708; 5,260,069.

Another approach to achieving sequential release would be to administer two or more populations of drug reservoirs, where the first population is designed to release a first agent at a certain rate and the second and subsequent populations are designed to release the second and subsequent agents at a different rate. Different rates of release are readily achieved by varying polymer composition, polymer thickness, particle size, in the case of polymer-based reservoirs; or, in the case of liposomal reservoirs, by lipid selection and vesicle size.

Example 4 describes the in vitro release of IL-12 from drug reservoirs composed of a biodegradable polymer, DL-lactide-co-glycolide. In brief, drug reservoirs containing IL-12 were placed in a vessle containing buffered saline and the release of IL-12 into the saline was measured as a function of time. Over a period of 11 days, 60% of the IL-12 was released.

As will be further described below, in use, any of the drug reservoirs described above are deposited as, for example, a small bolus into, at, or on the desired tissue site (see Examples 5 and 6). For example, a bolus of drug reservoirs can be deposited into the myocardium a few millimeters from the wall of the vessel where a depositing catheter is placed. The reservoirs described are capable of moving freely in the tissue through the interstitial space. Each reservoir is a focus point for angiogenesis, arteriogenesis, and muscle cell regeneration and can promote these processes repeatedly at different locations as it moves in the tissue. In this way, a relatively widespread therapeutic effect using a minimally invasive approach is achieved.

Also as described below, for use in the myocardium one option for delivery of the reservoirs is via the puncture of a small diameter needle through the wall of a coronary artery. Another option is direct injection into the myocardium during a surgical procedure from the pericardial aspect. An alternative route for direct injection into the myocardium is percutaneously (via a transvascular path) through the endocardium of the chambers of the heart. In skeletal muscle, reservoirs can be delivered through a small diameter percutaneous needle puncture. The reservoirs can also be delivered percutaneously during a PTCA, stent, or artherectomy procedure or by a percutaneous procedure solely for the placement of the reservoirs. In some instances it may be advantageous to place the reservoirs percutaneously, allowing more precise placement because of the simultaneous use of intravascular imaging techniques.

Another feature provided by the reservoirs of the present invention is mobility after deposition at a treatment site. Mobility is achieved, at least in part, by formulating the reservoirs to have a specific gravity less than that of interstitial body fluid. This will allow the reservoirs to float in the tissue fluid and move with the fluid.

### III. METHODS OF USING THE COMPOSITION

A. Method of Promoting Tissue Regeneration

In another aspect, the invention includes a method for promoting tissue regeneration in tissue post-damage or at risk of necrosis and damage. The method is contemplated for use as a primary treatment modality, i.e., not in conjunction with other drug, surgical, or interventional therapies. However, the methods may be employed in combination with such other treatment modalities. For example, the composition comprised of drug reservoirs containing one or more therapeutic agents, as described above, could be deposited after stent placement and balloon angioplasty to enhance blood perfusion to ischemic tissue surrounding the treated stenotic region. The composition could also be combined with delivery of other therapeutic agents used in treating coronary artery disease, such as anti-thrombotic agents. It is also contemplated to deposit the reservoirs at the time of surgery or during any minimally invasive procedure in or near the tissue of interest.

In one embodiment, tissue regeneration is accomplished by promoting tissue repair, angiogenesis, and/or arteriogenesis by depositing at a selected tissue site one or more drug reservoirs containing one or more therapeutic agents effective to (i) to attract circulating blood monocytes, other cells contributing to angiogenesis and arteriogenesis, or stem cells and their differentiated progeny cells to the tissue; (ii) to stimulate release of biological agents that promote angiogenesis, arteriogenesis, and infiltration of fully differentiated cardiac or skeletal muscle fibers (muscle cell regeneration); and to accomplish these phenomena by direct action on monocytes, stem cells, progenitors and other cells contributing angiogenesis, arteriogenesis, and muscle cell regeneration; and (iii) to influence the survival of circulating blood monocytes, derived macrophages other cells contributing to angiogenesis and arteriogenesis, stem cells and their differentiated progeny cells in such tissue. For example, drug reservoirs containing a chemoattractant such as MCP-1 and GM-CSF, which extends the survival of circulating blood monocytes and derived macrophages, are deposited at an at-risk ischemic site or at a site post ischemia. The MCP-1 serves to attract circulating blood monocytes to the tissue area and to stimulate the attracted monocytes/derived macrophages to release factors involved in angiogenesis and/or arteriogenesis. Attraction and stimulation of peripheral monocytes, and subsequent migration of the monocytes into the interstitial space for maturation into tissue macrophages, initiates release of signaling factors that directly influence formation of capillaries and remodeling of arterioles into arteries. The presence of GM-CSF enhances the survival time of the macrophages, extending the period of release of the biological factors from the recruited macrophages. In this way, angiogenesis and/or arteriogenesis in tissue surrounding the target site is promoted.

For example, drug reservoirs containing a chemoattractant, such as stem cell factor (SCF), which attracts circulating stem cells into the tissue, and GM-CSF, which has the ability to increase the number of circulating stem cells, are deposited at an at-risk ischemic site or at a site post ischemia. The SCF serves to attract circulating and resident stem cells to the tissue area and GM-CSF serves to increase the number of stem cells entering the area. Growth factors facilitate differentiation of stem cells to myoblasts, which fuse to regenerate cardiac and skeletal muscle tissue. In this way, regenerated muscle cells in tissue surrounding the target site is promoted.

Example 5 describes use of drug reservoirs containing GM-CSF, MCP-1, and RANTES for cell and tissue regeneration in rabbits subsequent to an ischemic episode. GM-CSF, MCP-1, and RANTES each have activity as chemoattractants for cells involved in cellular regeneration and as stimulants for differentiation and proliferation of these cells.

By way of another example, the drug reservoirs can also be formulated to include a cellular adhesion molecule, such as ICAM, to promote adhesion of monocytes or other angiogenic, and arteriogenic cells to the tissue region. The cellular adhesion molecule can be coupled to the outer surface of one or more drug reservoirs, or contained within the reservoirs for release. The adhesion molecules serve as ligands for integrins and similar species present on the plasma membranes of macrophages, fibroblasts, and smooth muscle cells. The adhesion molecules also attract macrophages to the drug reservoirs and enable physical binding to the reservoirs to secure the macrophages in the desired region and maximize exposure of the macrophages to the molecular signaling factors released from the drug reservoirs.

In addition to a chemoattractant, a stimulation agent, and a survival-promoting agent, the drug reservoirs can optionally contain other agents involved in the angiogenesis, arteriogenesis, and muscle cell regeneration process. For example, it may be desirable to include a growth factor or a cytokine in the drug reservoirs, to participate in the cascade of events in the angiogenesis, arteriogenesis, and muscle cell regeneration process or to stimulate or alter certain events in the process.

As noted above, the release kinetics of the one or more entrapped therapeutic agents can be controlled through selection and formulation of the drug reservoirs. The drug reservoirs serve to attract, retain, stimulate, and enhance survival, proliferation and differentation of progenitor cells, including but not limited to peripheral monocytes, cardiac myocytes, and skeletal myoblasts, which then release the various signaling factors involved in the tissue regeneration, which may or may not include angiogenesis and/or arteriogenesis. Because the drug reservoirs release the biological agents over a controlled length of time, a sustained local concentration of the agents is maintained for the duration of the target tissue response. Also, multiple biological agents with different target cell responses can be released with different kinetic profiles, resulting in effective concentrations of the required agents over the duration of the tissue response. In this way, various cell populations involved in angiogenesis, arteriogenesis, and muscle cell regeneration can be coordinated and harmonized to promote the formation of functional new vasculature, cardiac and peripheral muscle in the tissue at risk. For example, stimulation of proliferation of vascular endothelial cells that line the lumen of a new arteriole or small artery can be coordinated with stimulation of proliferation and motility of vascular smooth muscle cells for formation of the muscular layer covering the vascular endothelial cells. In one embodiment of the invention, the deposited drug reservoirs serve to attract, stimulate, and promote the survival of monocytes, and upon stimulation, the monocytes release the appropriate factors at the appropriate time in the angiogenesis and/or arteriogenesis process. In another embodiment, the drug reservoirs additionally contain agents that supplement the concentration of factors released from the recruited monocytes, for participation in and enhancement of the formation of new arterioles.

In another embodiment, the invention contemplates a method of regenerating nerves during the regeneration of muscle tissue. Deposition of drug reservoirs loaded with appropriate therapeutic and biological agents to stimulate nerve growth in a post-ischemic area is contemplated.

In the method of treatment, the drug reservoirs can be deposited at a tissue site by various techniques. For example, the reservoirs can be injected directly into tissue or into a cavity adjacent to the target tissue using an infusion needle. The reservoirs can be surgically implanted in neat form or in combination with a medical device, such as a stent.

The drug reservoirs can also be deposited at a tissue site by applying a thin layer of a viscous paste, hydrogel, or other polymeric carrier matrix containing the reservoirs to an endoluminal wall adjacent to the target site, or other body cavity adjacent to the target tissue, such as the pericardial sac surrounding the epicardial surface of the heart. The polymeric carrier forming the paste or gel can be biodegradable and/or serve as a temporary wall support while the biological agents are released. Endoluminal paving systems have been described, for example, in U.S. Pat. No. 5,328,471. A specific example of an endoluminal paving device designed for the present composition is described below.

By way of example, drug reservoirs formulated into a paste can be deposited in the pericardium or in the cerebrospinal fluid space for induction of angiogenesis and arteriogenesis in regions of brain infarct or ischemia. The concentration gradients established by the reservoirs would attract angiogenic and arteriogenic cells from the bloodstream into the tissue. Alternatively, the paste or a gel can be applied to the wall of a vessel in the proximity of a vessel restriction (blockage) or in a healthy vessel region to achieve transport through the vessel wall to a site of actual or potential ischemia.

The reservoirs can also be deposited using a catheter, as will be described in more detail below. Catheters commonly known in the art, such as catheters capable of direct infusion of drugs and balloon catheters having the capability of administering a therapeutic agent, are suitable. In a preferred embodiment, a catheter having a structure that penetrates into the tissue or vessel wall for placement of the reservoirs in the interstitial space is used. For example, a device with small needles for delivery of angiogenic factors is described in U.S. 6,152,141, as is incorporated by reference herein. Another exemplary device is described below.

The drug reservoirs used in the methods of the present invention can also be incorporated into conventional pharmaceutical compositions for transmural delivery. In the case of continuous catheter delivery, the drug reservoirs will be incorporated into an acceptable fluid carrier, e.g., formulated with sterile water, isotonic saline, glucose solution, or the like. The formulations may contain pharmaceutically acceptable auxiliary substances as are generally used in pharmaceutical preparations, including buffering agents, tonicity adjusting agents, such as sodium acetate, sodium lactate, sodium chloride, potassium chloride, and calcium chloride, and the like. General methods for preparing such pharmaceutical formulations are described in Remington's PHARMACEUTICAL SCIENCES, Mack Publishing Co., Philadelphia, Pa., 1985.

The composition is delivered for a time sufficient to achieve the desired physiological effect, i.e., the promotion of blood vessel growth and muscle cell repopulation in tissue surrounding the target site. Generally, the total amount of the biological factors delivered will vary according to the condition of the tissue, patient characteristics, and desired effect. Determination of the appropriate dose regimen for a given patient is well within the skill of the attending physician. Since the proper dose varies from person to person based on the age and general state of health, it is a common practice of physicians to “dose-titrate” the patient; that is, to start the patient on a dosing regimen which is at a level below that required to produce the desired response, and gradually increase the dose until the desired effect is achieved.

It will also be appreciated that the formulation containing the drug reservoirs may include other agents in addition to the selected biological factor(s). For example, the formulations may include anti-coagulants and anti-thrombotic agents, such as heparin, low molecular weight heparin, and the like.

B. Method of Promoting Cellular Immune Response for Cancer Therapy

In another aspect, the invention includes a method of promoting a cellular immune response to cancer in a subject. The method comprises depositing in or adjacent to a primary tumor or metastasis a composition containing one or more agents effective to (i) attract one or more of lymphocytes, macrophages, dendritic cells, natural killer cells, progenitor cells, and accessory cells to the tissue site; (ii) stimulate direct action on local and attracted cells and components of the extracellular matrix, and the release of biological agents from said local and attracted cells that promote cytotoxicity to cancerous cells; and (iii) influence the survival of the attracted and locally regenerated cells in such tissue, to promote a long term immunogenic response to the cancerous cells.

The composition comprised of drug reservoirs containing one or more therapeutic agents, as described above, is deposited via injection directly into a primary tumor or via catheter into a remote site infected with tumor cells. The composition can be employed in combination with delivery of other chemotherapeutic agents used in treating cancer. It is also contemplated to deposit the reservoirs at the time of surgery or during any minimally invasive procedure in or near the tissue of interest. The preparations described above are equally suitable for this aspect.

Example 6 describes use of a composition composition comprised of biodegradable polymeric drug reservoirs containing IL-12 for regression of tumors in mice. As described above, IL-12 acts as a chemoattractant for lymphocytes, macrophages, antigen presenting cells (including dendritic cells), and inflammatory cells. IL-12 also acts as a stimulant for these cells, promoting proliferation, differentiation, and or antigen presentation. These activities result in a response that ultimately results in tumor regression.

### IV. DEVICES FOR ADMINISTRATION OF EXEMPLARY COMPOSITIONS

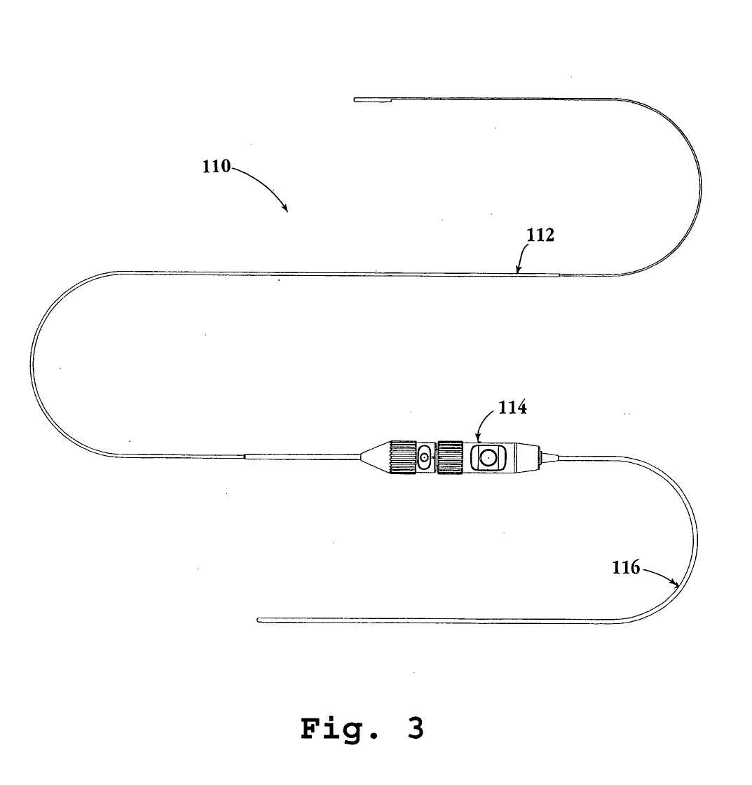

In another aspect, the invention provides a device for depositing the drug reservoirs at a selected tissue site. Various exemplary embodiments of the device will now be described with respect to FIGS. 3-18.

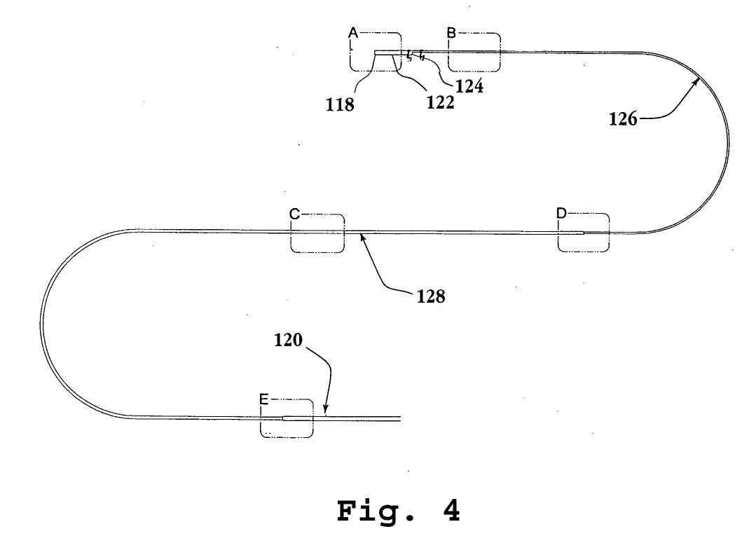

FIG. 3 is a plan view of an exemplary device for use in depositing drug reservoirs described above. Device 110 is comprised of a catheter shaft 112, a user interface 114, and a sheath for depositing piston 116. Catheter shaft 112 is shown in detail in FIG. 4, where the shaft extends between a distal tip 118 and a reinforced shaft segment 120. The reinforced shaft segment is adjacent to user interface 14 (FIG. 3) and designed to prevent kinking of the shaft during use, as later described. The catheter shaft further includes a monorail guidewire engagement segment 122 having a proximal guidewire port 124 for introduction of a guidewire into segment 122. A distal flexible shaft segment 126 provides for navigation in tortuous vessels, with a more rigid proximal shaft segment 128 to allow for pushing and torquing of the distal shaft segment.

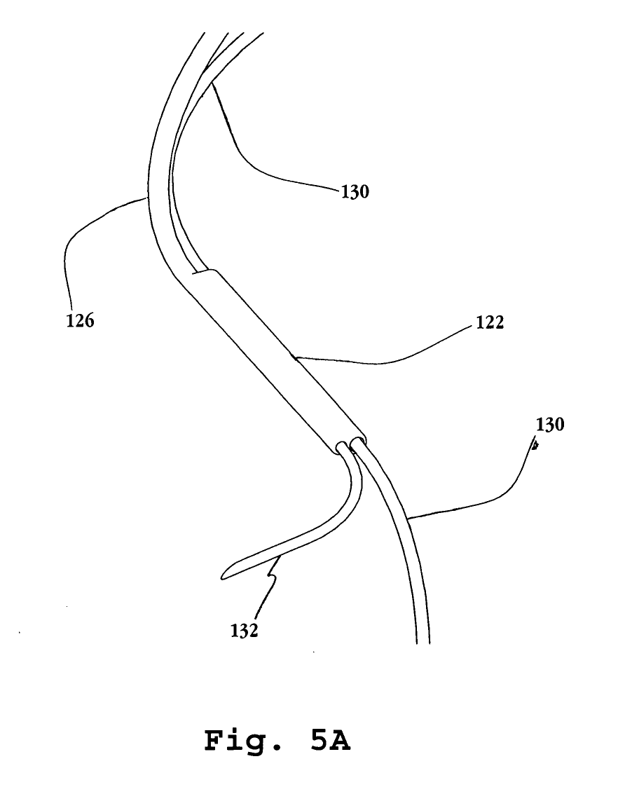

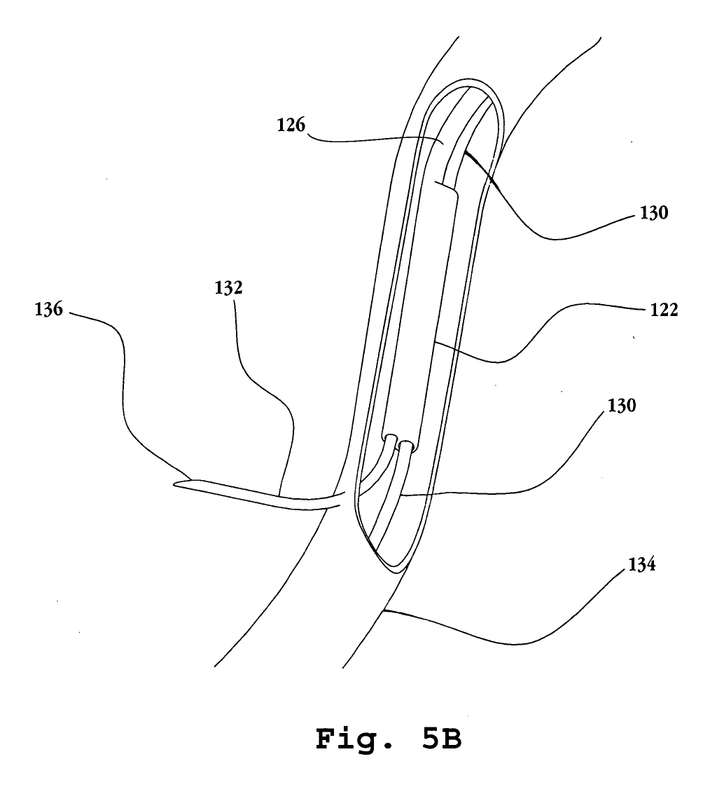

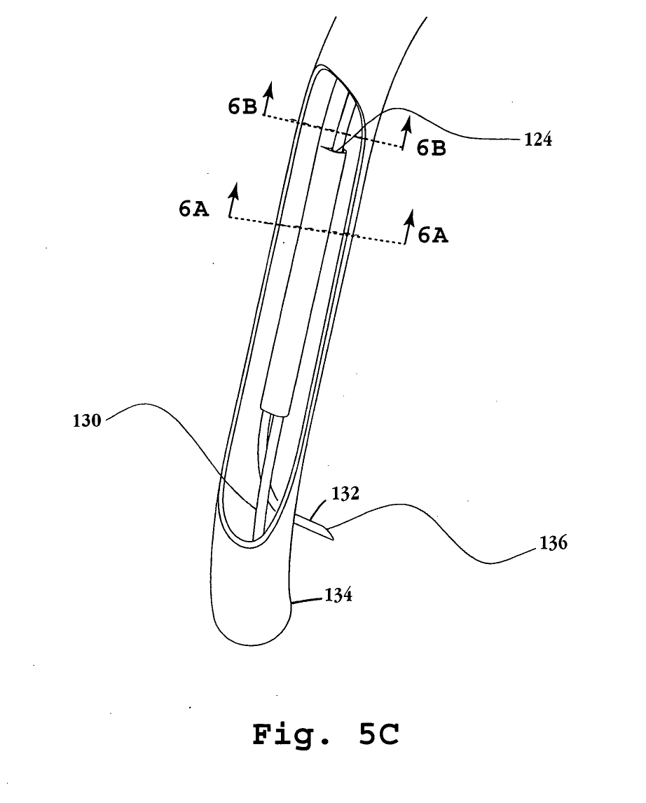

A detail view of a distal segment of catheter shaft 112 is shown in FIG. 5A. Distal flexible shaft segment 126 and the monorail guidewire engagement segment 122 of the catheter shaft are shown. The catheter shaft rides on a guidewire 130 through engagement only at the monorail segment. The limited engagement between the guidewire and the shaft maximizes steering of the distal tip through tortuosities in small caliber vessels. The shaft terminates in a needle 132 for depositing drug reservoirs at a tissue site. In one embodiment, as illustrated in FIGS. 5B and 5C, the needle punctures the tissue for deposition in the interstitial space. FIG. 5B shows the distal flexible catheter shaft segment 126 in position in a body vessel 134. The shaft engages guidewire 130 at the monorail guidewire segment 122. Needle 132 terminates in a beveled tip 136 capable of piercing the vessel wall for entry into the interstitial space and placement of drug reservoirs. FIG. 5C shows the same device positioned in vessel 134 but where beveled needle 132 is rotated for puncture of the vessel wall at another position from that illustrated in FIG. 5B.

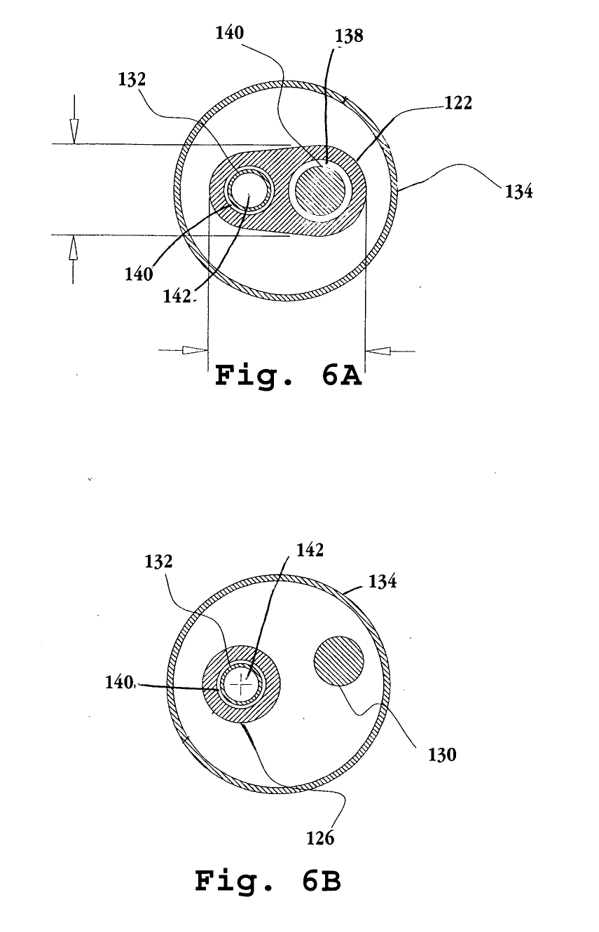

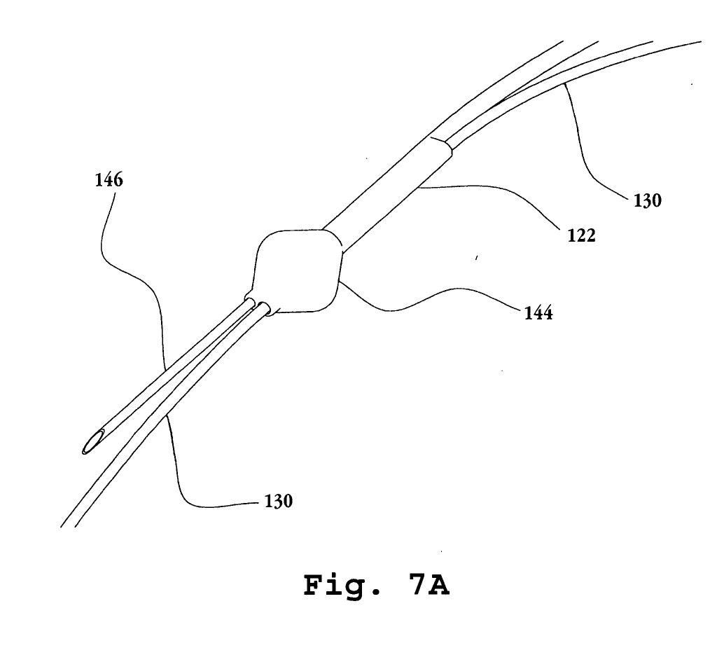

FIGS. 6A-6B are transverse cross-sectional views through lines 6A-6A and 6B-6B in FIG. 5C showing in cross section the monorail segment 122 (FIG. 6A) and a region just proximal to the monorail segment (FIG. 6B). In FIG. 6A, monorail segment 122 is positioned in vessel 134. Guidewire 130 is engaged with the monorail segment and positioned in guidewire lumen 138. Shaft lumen 140 of the catheter shaft is visible, and disposed within shaft lumen 140 is needle 132 defining a needle lumen 142 for delivery of the drug reservoirs. In FIG. 6B, the region proximal to the monorail guidewire segment is shown, where the distal shaft segment 126 defining shaft lumen 140 is seen. Needle 132 is disposed in shaft lumen 140 and defines needle lumen 142. Guidewire 130 at this position in the device is not engagingly coupled to the catheter shaft.

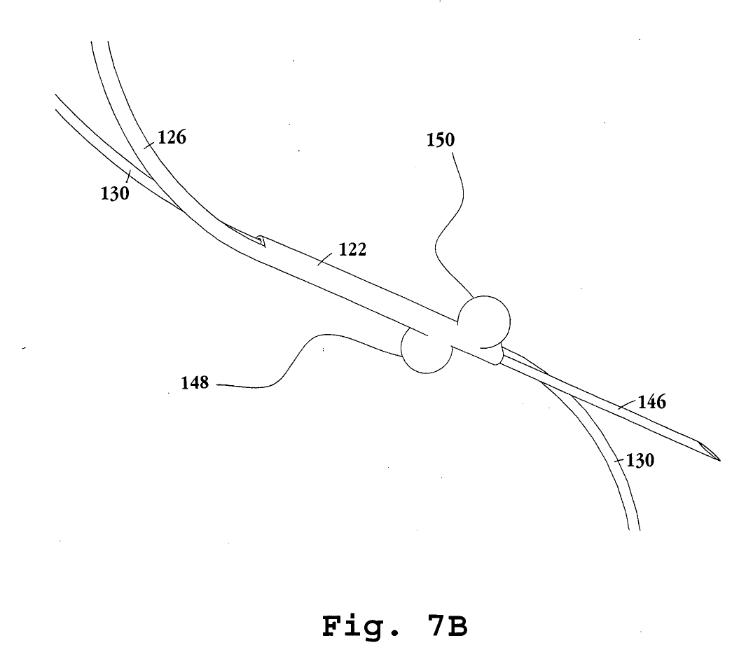

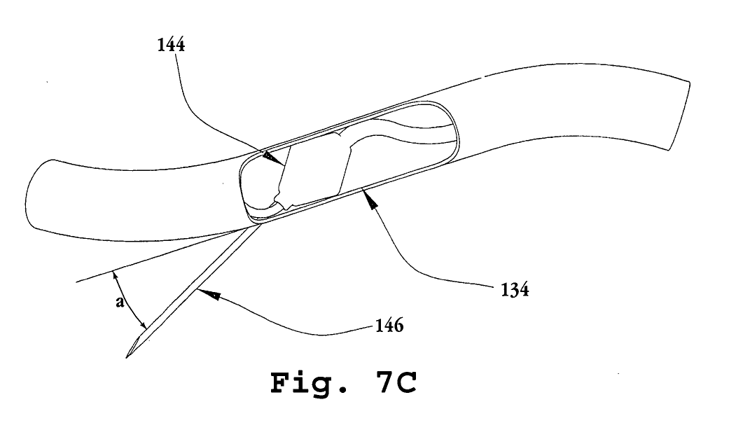

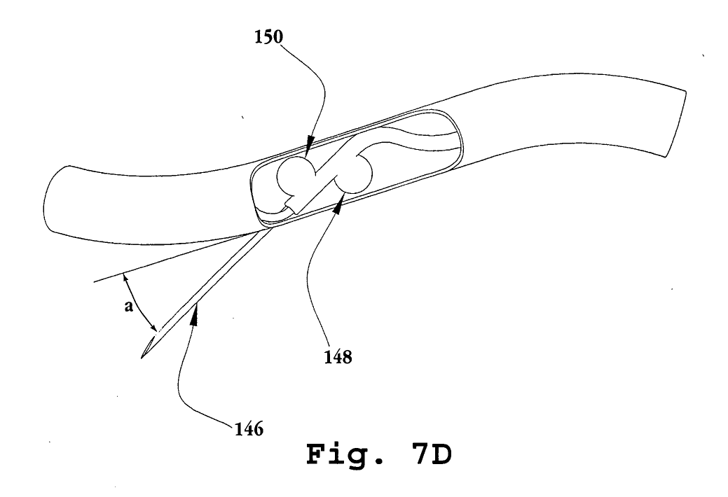

FIGS. 7A-7B illustrate another embodiment of the device where a distal balloon is used to position the catheter tip in the form of a straight needle for tissue penetration. In FIG. 7A guidewire 130 is shown engaged with monorail segment 122 distal to the flexible shaft segment 126 of the device. Positioned on the distal portion of the monorail segment is a balloon 144 having a biconical profile when inflated (as shown). As can be appreciated, inflation of the balloon positions the catheter distal tip at an acute angle to the vessel wall, allowing a straight needle 146 to puncture the vessel wall for deposition of drug reservoirs in the adjacent tissue space. FIG. 7B shows an alternative embodiment where two spherical balloons 148, 150 are positioned on opposing sides of monorail segment 122. The balloons, shown in an inflated condition, position the catheter distal tip at an acute angle to the vessel wall, allowing straight needle 146 to puncture the vessel wall.

FIGS. 7C and 7D show the two above embodiments, with biconical and double spherical balloons (respectively) disposed in a blood vessel. In FIG. 7C, it can be appreciated that the vessel wall 134 constrains the balloon 144 so that needle 146 pierces the vessel wall at angle α. In FIG. 7D, the two spherical balloons 148, 150 are shown to be constrained by the vessel wall so that needle 146 pierces the vessel wall at angle α.

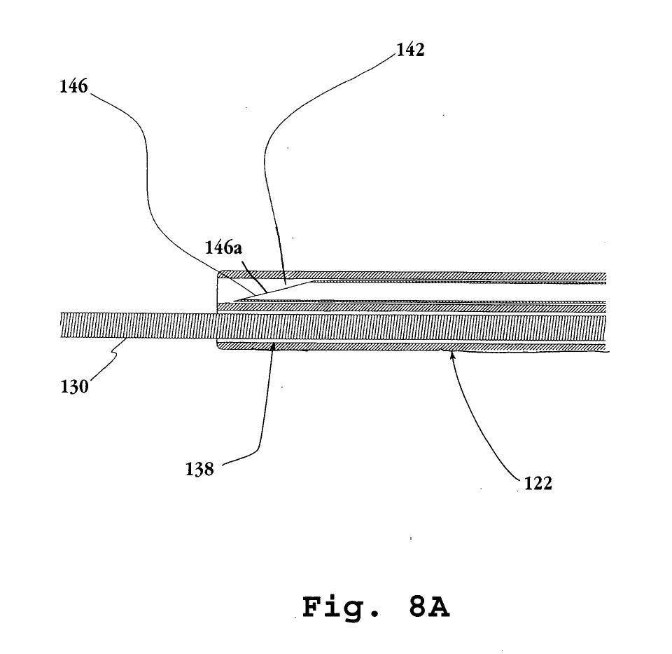

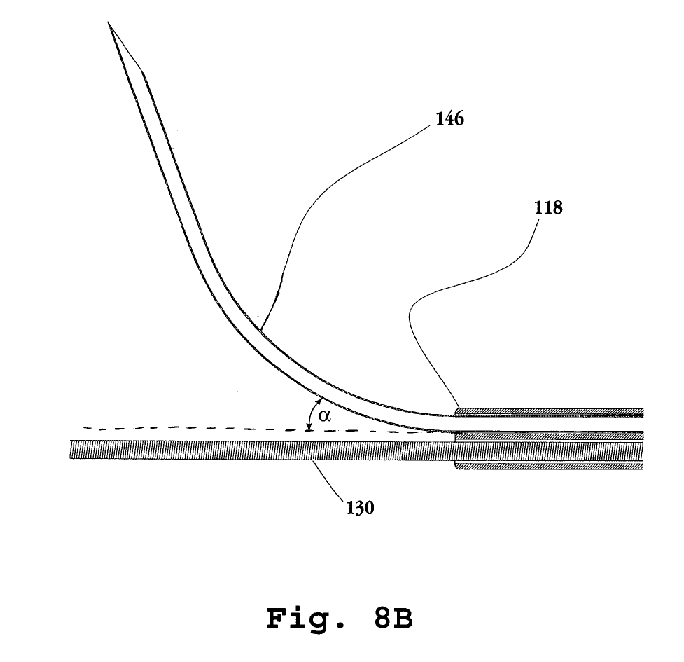

FIG. 8A is a cross-sectional view of region A in FIG. 4. Monorail segment 122 includes guidewire lumen 138 and shaft lumen 142. Positioned in shaft lumen 142 is needle 146 having a beveled tip 46a. Guidewire 130 is disposed within lumen 138. FIG. 8B shows the same cross-sectional view, but with needle 146 extended from the catheter shaft, beyond the distal tip 118, for placement of drug reservoirs at a desired tissue site. The needle is illustrated in a deployed state with an angle α. It will be appreciated that the angle can be widely varied depending on the preformed curvature of the needle itself as well as the angle of deployment achieved through device means such as balloons, as discussed in FIGS. 7A-7D.

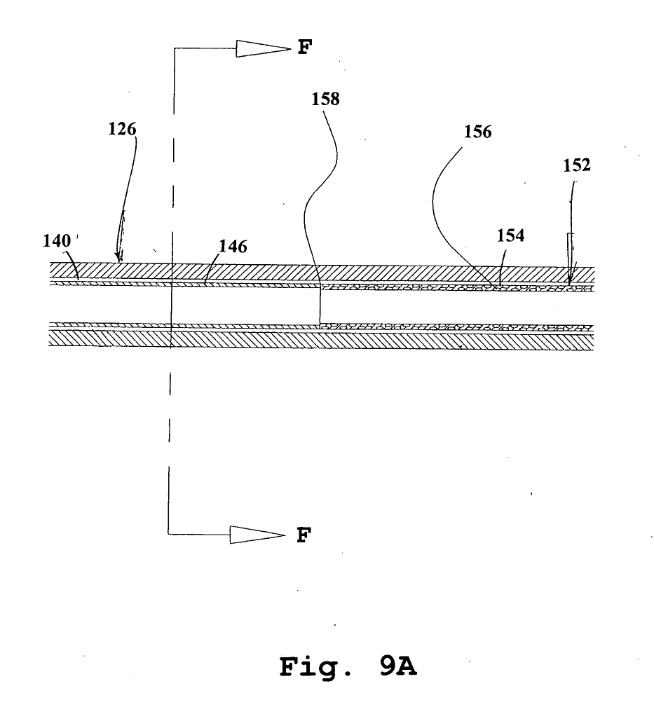

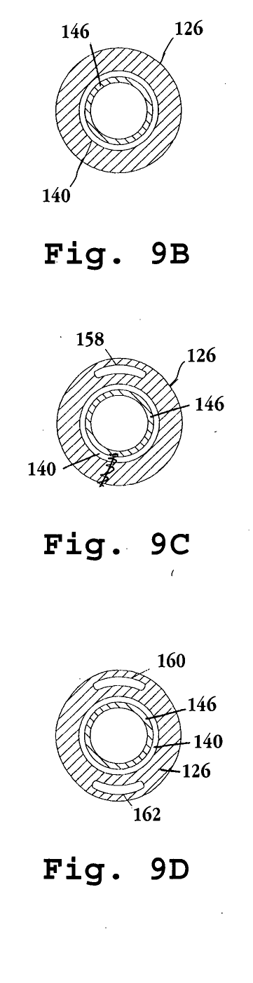

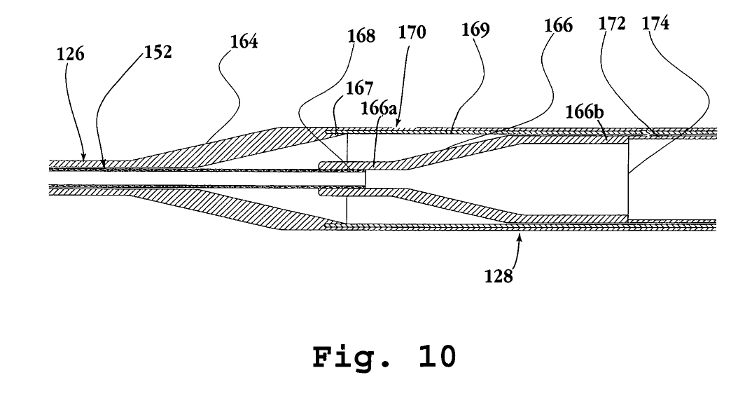

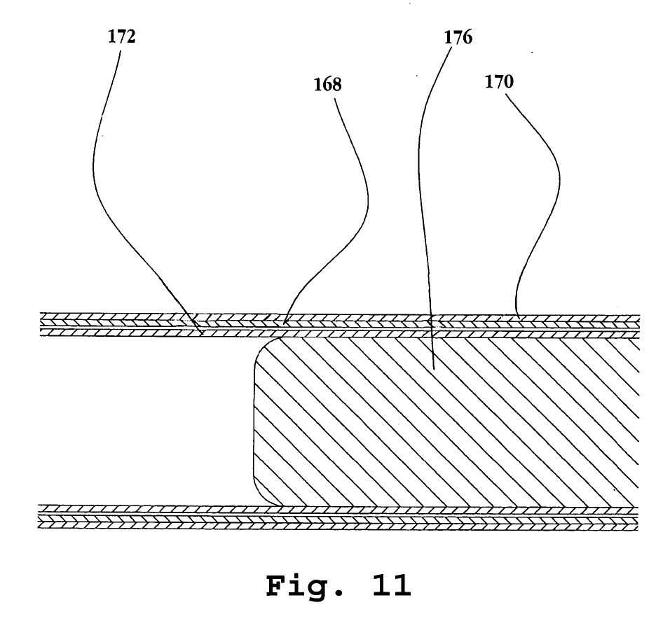

FIG. 9A is a cross-sectional view of region B in FIG. 4. Distal, flexible catheter shaft segment 126 defines shaft lumen 140 in which needle 146 is positioned. Abutting needle 146 is a composite tube 152 comprised of a thermoplastic elastomer 154 and a wire braid 156. Interface 158 between the composite tube and the needle serves as a point of contact for these two components, and an adhesive material or other attaching means can be positioned at the interface.

FIG. 9B is a cross-sectional view through line F-F in FIG. 9A, where needle 146 disposed in lumen 140 of the catheter shaft 126 is seen. FIGS. 9C and 9D are cross-sections at the same position, but in devices with a biconical balloon (described in FIG. 7A) or with two spherical balloons (described in FIG. 7B). Devices that include one or more balloons will have one or more passages, such as passage 158 in FIG. 9C or passages 160, 162 in FIG. 9D, for introduction of a fluid, typically angiographic contrast medium, into the balloon for inflation and for removal of fluid for deflation.

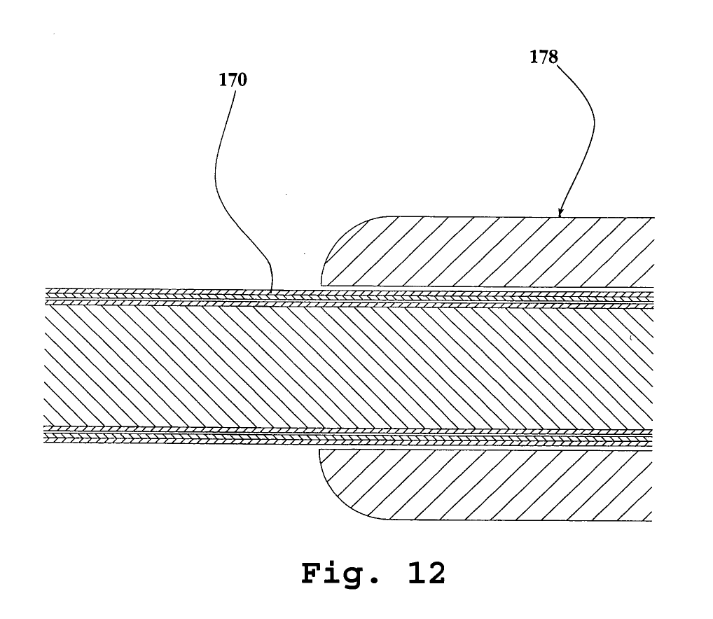

FIG. 10 is a longitudinal cross-section of Area D in FIG. 4, over the transition region 164 from the distal flexible shaft segment 126 of the catheter and the more rigid proximal shaft segment 128. Transition region 164 consists of a fused, thermoformed, thermoplastic elastomer spanning the region between the flexible distal shaft and the rigid proximal shaft. Composite tube 152 positioned in the lumen of the flexible shaft connects to an adaptor 166, formed preferably of a metal such as stainless steel. The composite tube and the connector at a first end 166a are joined at interface 168 by any suitable means, such as adhesive bonding, solder, welding. Rigidity in the proximal shaft region 128 is achieved, for example, by incorporation of a reinforcing hypotube 169 along the inner wall of the elastomeric tubular wall 170 forming the proximal shaft 128. In general, hypotube 169 and the proximal shaft segment are joined and fixed at interface 167 by thermoforming the thermoplastic elastomer component upon the hypotube, by adhesive bonding, or by any other suitable means that allows flexibility of the elastomer-hypotube composite structure.

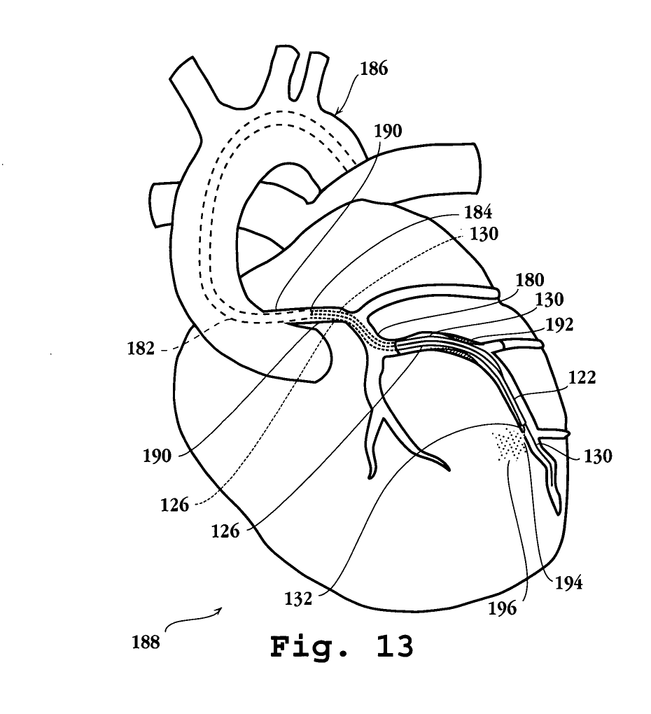

Abutting the adapter at a second end 166b is a hypotube 172. Hypotube 172 is disposed within the lumen defined by hypotube 169 and functions as a barrel of a reservoir injecting syringe that is filled with the drug reservoir formulation to be deposited at the tissue site. For example, the hypotube can be filled with lyophilized reservoir material or with a liquid reservoir suspension for displacement by a piston through the composite tube, into the needle lumen and ultimately into the desired tissue. This aspect of the device is illustrated in more detail in FIG. 11.

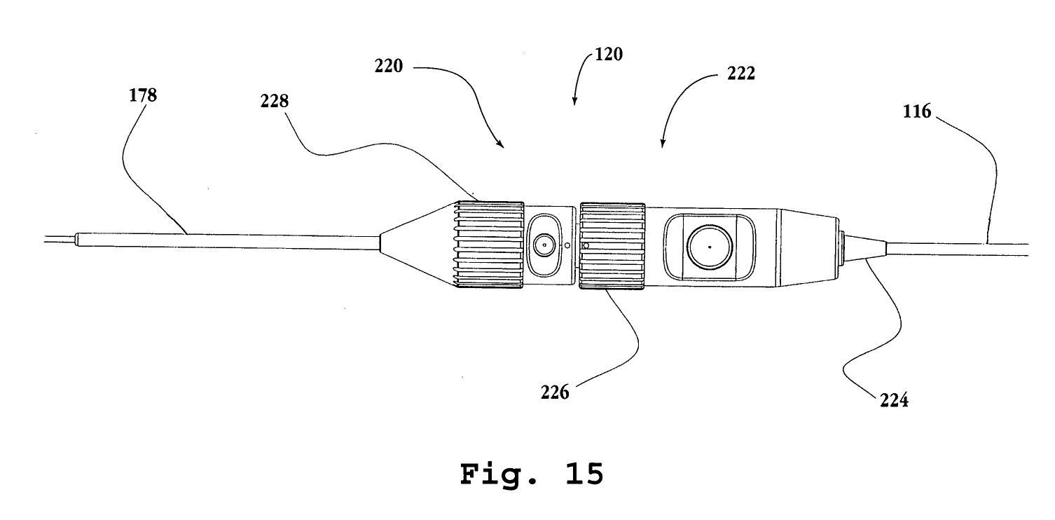

FIG. 11 is a longitudinal cross-section of Area C in FIG. 4 showing the position of movable piston 176 within hypotube 172. Piston 176 is movable by a mechanical mechanism in the user interface, described in FIGS. 15-17 below, and is a continuous metal mandrel that slides through the hypotube. Piston 176 can be formed of stainless steel and coated with polytetrafluoroethylene at the distal tip to serve as a lubricant to allow the piston to slide freely. The piston slides through the hypotube and preferably creates a liquid-tight seal to achieve displacement of the drug reservoir formulation. The polytetrafluorethylene coating also acts as a compliant sleeve to ensure a liquid-tight seal.

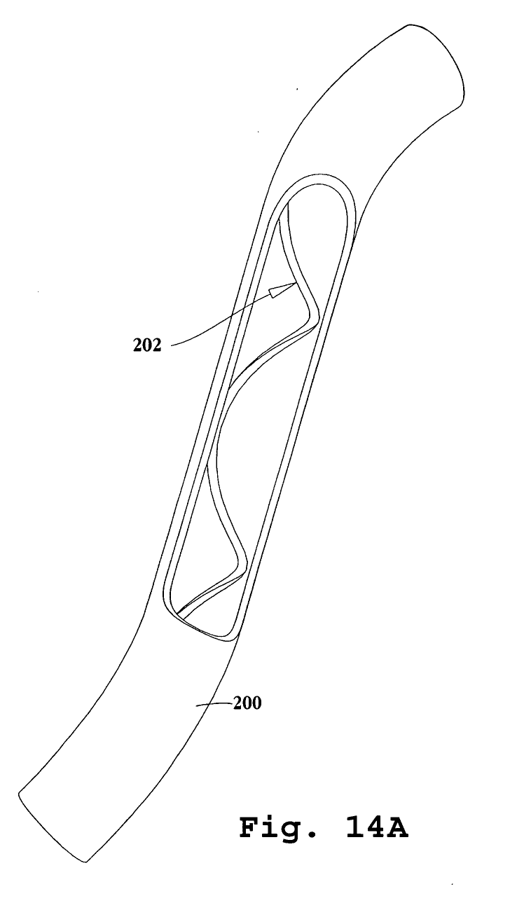

FIG. 12 is a longitudinal cross-section of Area E in FIG. 4 showing the reinforced shaft segment 120 (FIG. 4) just distal to the user interface 114 (FIG. 3). An extruded tubular member 178 is anchored in the user interface and serves to stiffen the shaft for better control over positioning and placement of the device. In particular, the added reinforcement and stiffening provided by member 178 eliminates kinking of the shaft at this region of torque and flexure.

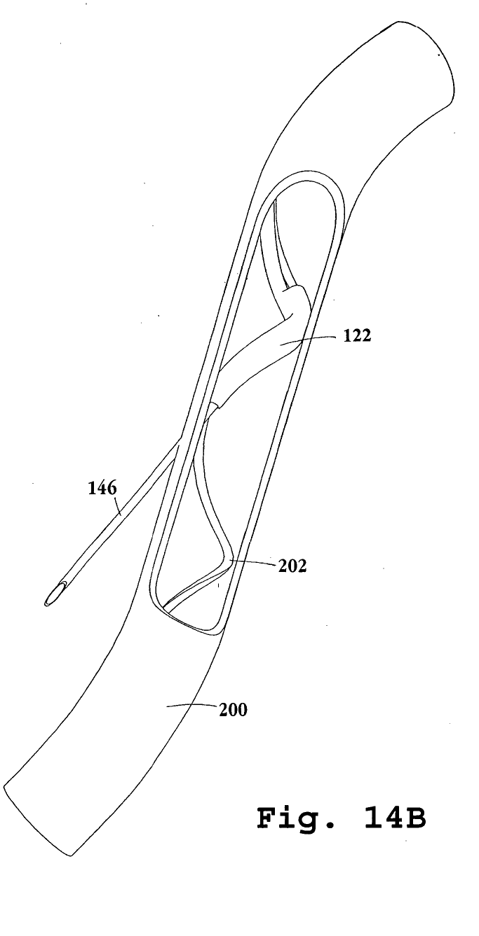

Before describing the user interface in detail, an exemplary illustration of the device for deposition of drug reservoirs into, for example, the myocardium at an ischemic site distal to an atherosclerotic occlusion producing the ischemia is provided in FIG. 13. Seen in the figure is the distal flexible shaft portion 126 of the device 110 positioned in the left anterior descending coronary artery 180. The device is introduced by means of a coronary artery guiding catheter 182 positioned in the left main coronary artery 184. The aortic arch, inferior vena cava, and ostium of left main coronary artery are shown as 186, 188, 190, respectively. The device is guided by coronary guidewire 130 past an atheromatous occlusion 192 to an ischemic site or a site at risk of ischemia 194. Needle 132 is deployed from the device and a bolus 196 of drug reservoir formulation is deposited at the ischemic site.

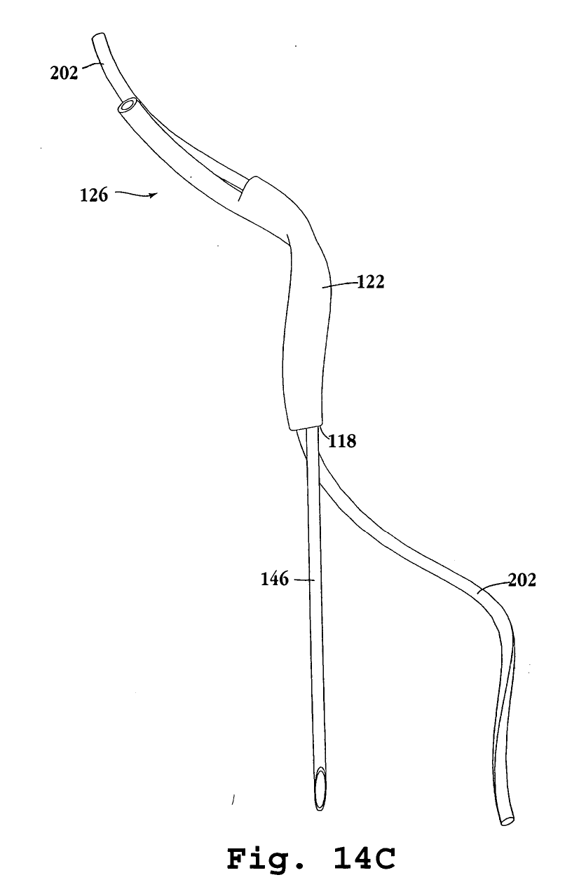

In the device embodiments described above, a conventional, straight guidewire (element 130) was used to guide the device to the desired tissue site. In an alternative embodiment, shown in FIGS. 14A-14C a helical guidewire is used for advancing the device and positioning the needle for tissue penetration. FIG. 14A shows a vessel 200 and a helical guidewire 202 placed in the vessel lumen. A catheter device in accord with this aspect of the invention is inserted into the vessel by tracking over the helical guidewire, as illustrated in FIG. 14B. For viewing simplicity, FIG. 14C omits the vessel to show the catheter device at its flexible, distal end, engagingly coupled at the monorail segment 122 with helical guidewire 202. Extending from distal tip 118 of the catheter shaft is a straight needle 146, which, as seen in FIG. 14B, is angled by virtue of the helical guidewire for penetration of the vessel wall. It will be appreciated that the angle of the turns in the helical guidewire can be varied to vary the angle of penetration with the vessel wall.

The user interface 120 shown in FIG. 4 will now be described with reference to FIGS. 15-17. FIG. 15 shows an overall top view of a preferred embodiment of user interface 120. The interface is comprised of a distal segment 220 that is rigidly coupled to the catheter shaft at the extruded tubular member 178 (described in FIG. 12). At the opposing end of the user interface is a proximal segment 222 that is rigidly coupled internally to-the hypotube (element 172, FIG. 10), composite tube (element 152, FIG. 9A), and needle, collectively referred to as the “needle assembly”, and permits user control of the needle, including deployment or extension from the catheter shaft, withdrawal of the needle into a retracted position inside the shaft, and needle rotation. Proximal segment 222 also permits control of delivery of the drug reservoir formulation by advancing the sliding piston (element 76, FIG. 11). Coupled to the proximal segment of the user interface is grommet 224 that provides strain relief for piston sheath 116 (also described in FIG. 4). Grooved grip regions 226, 228 on the proximal and distal segments of the user interface, respectively, are movable by the user for control of the device. Specifically, rotation of grip region 228 allows for application of torque to the catheter. Movement of grip region 226 provides control of the needle assembly and rotation of the needle.

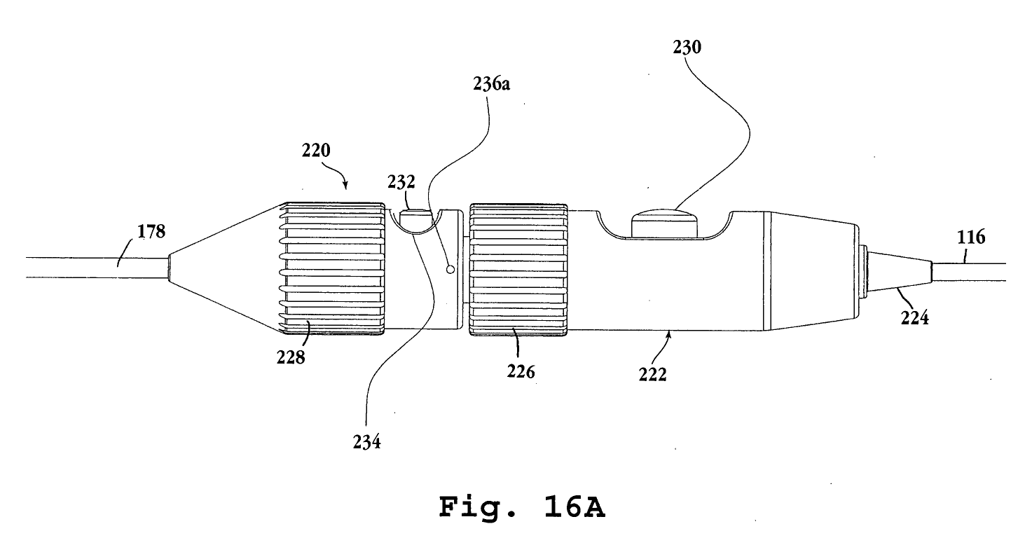

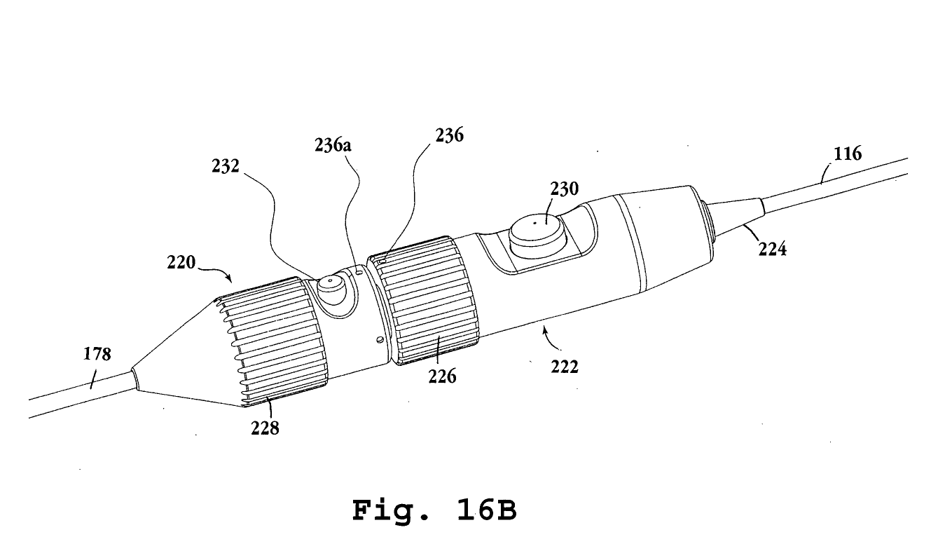

FIGS. 16A and 16B are side (FIG. 16A) and oblique (FIG. 16B) views of the user interface shown in FIG. 15. Depositing actuator button 230 disposed in proximal segment 222 of the user interface is moveable by the user to advance piston 176 (not visible in the figure, refer to element 76 in FIG. 11). Depression of the depositing actuator button engages a pair of rollers or a sliding collet to advance the piston and dispense a reproducible deposit of drug reservoir formulation. On the distal segment of the user interface a needle assembly lock actuator button 232 is moveable by the user. Depression of button 232 unlocks proximal segment 222 allowing it to be rotated, advanced, or retracted for corresponding rotation, advancement, or retraction of the needle. Button 232 in its fully extended position, e.g., when not depressed, locks the proximal segment and needle assembly in position with respect to the distal segment and the catheter shaft. Needle assembly lock actuator button 232 sits in a recess 234 to prevent inadvertent unlocking of the needle assembly. An index indicator 236a, 236b, is provided on the distal and proximal segments to show the relative extent of rotation of the needle and the direction of the angled distal needle segment with respect to the catheter shaft.

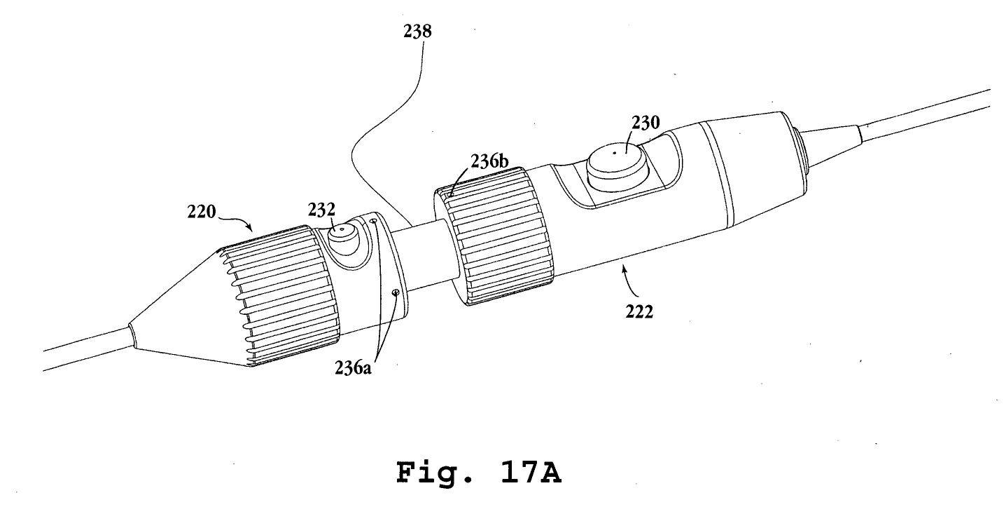

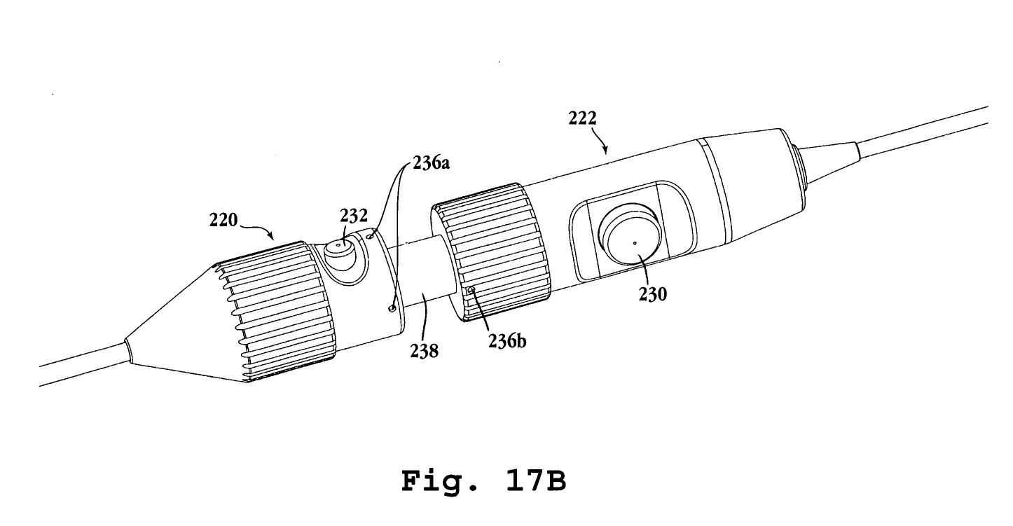

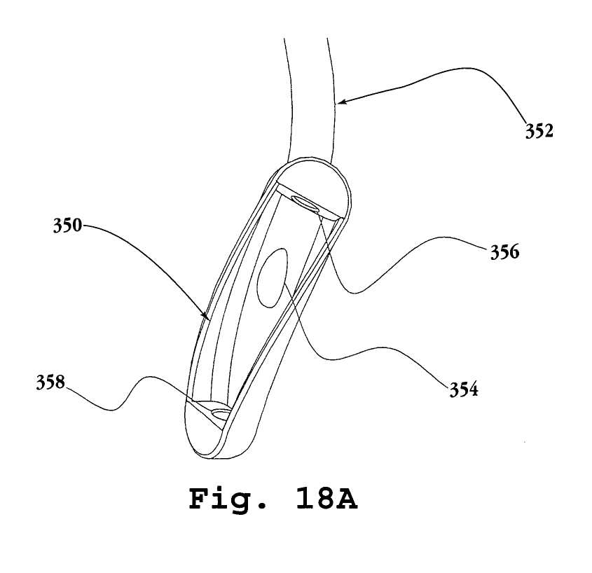

FIGS. 17A-17B are oblique views of the user interface of FIGS. 15-16 showing the proximal segment in its withdrawn position (FIG. 17A) and in its withdrawn position and rotated 900 (FIG. 17B). The proximal segment in its withdrawn position corresponds to a full retraction of the needle within the distal segment of the catheter shaft. In previous figures, the user interface is shown in a position where the needle assembly is extended, with the needle deployed for deposition of drug reservoir formulations at the tissue site. A needle body assembly 238 is rigidly attached to the proximal segment, and is slidably moveable and rotatable within the distal segment. Rotation of the proximal segment, as shown in FIG. 17B, corresponds to rotation of the needle. The extent of rotation is visualized using index indicators 236a, 236b.

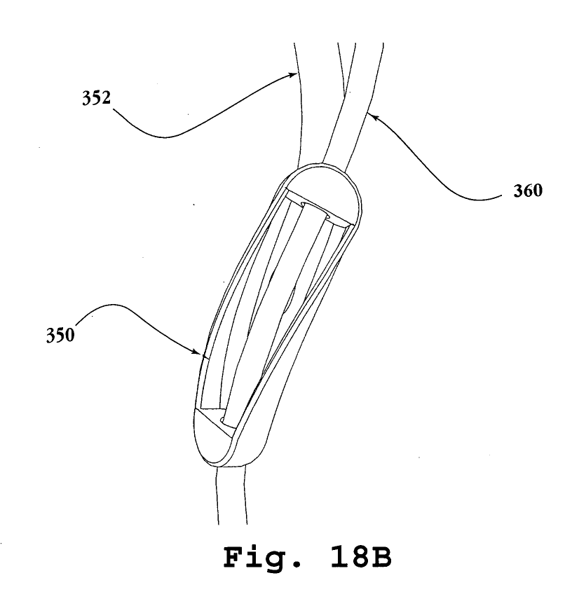

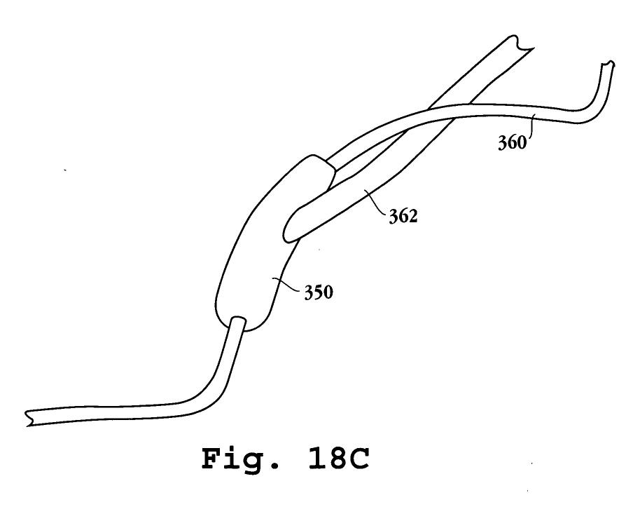

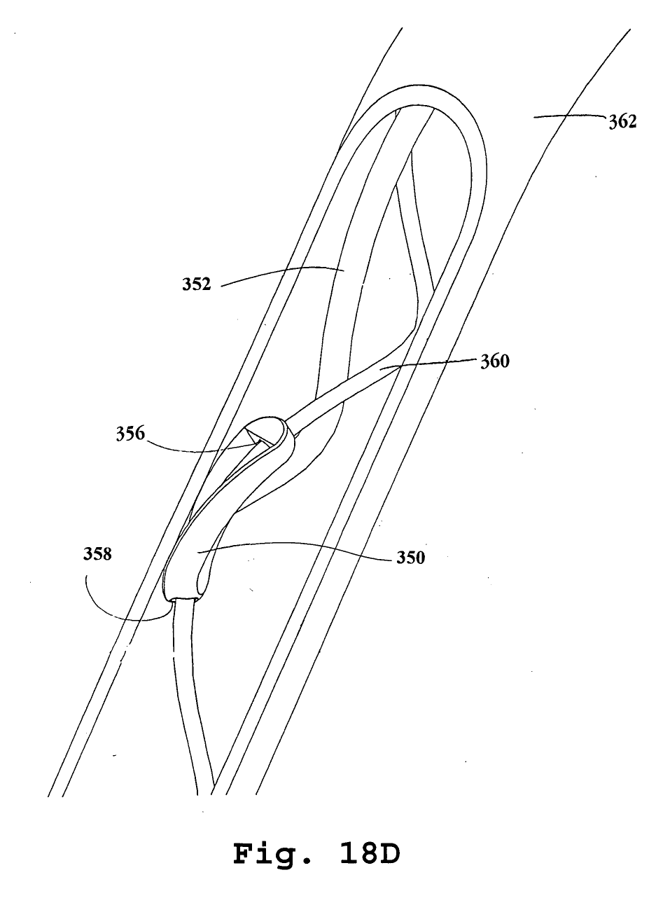

FIGS. 18A-18D illustrate an endoluminal paving device for deposition of the composition along the wall of a vessel or cavity in the body. The device includes a somewhat elongate flexible cup 350 with a central cavity for receiving the composition via a supply tube 352. The supply tube is preferably integrally formed with the flexible cup and terminates at opening 354, as seen best in FIG. 18A, through which the composition is passed from the supply tube into the flexible cup. The cup has an open face for deposition of the composition along the vessel wall and closed back portion (see FIG. 18C) and side walls to ensure deposition along the wall.

Additional openings 356, 358 are provided in the cup for introduction of a guidewire 360 into and out of the cup. A helical guidewire 360 is seen inserted into the upper opening 356 and the lower opening 358 of the cup in FIGS. 18B (front view), 18C (a back view), and 18D (inserted into a vessel). Suitable sealing mechanisms, if needed, can be provided at the junction of the guidewire at ports 356, 358 to eliminate or reduce loss of composition at the junction.

As seen best in FIG. 18D, the helical guidewire is in rigid contact with the inner wall of vessel 362. In use, the flexible cup, which serves as a monorail segment, is physically moved along the helical guidewire by user or automatic control at the proximal end. As the cup travels along the monorail, the composition is introduced into the open cup which is in contact with the inner vessel wall. Movement of the cup spreads a layer of the composition on the vessel wall essentially “paving” the vessel wall with the composition. Supply of the reservoir composition for paving is accomplished by piston displacement of the reservoir preparation as described previously. Movement of paving device along its path is accompanied by constant displacement and extrusion of the reservoir preparation into the cup.

From the foregoing, it can be seen how various objects and features of the invention are met. A composition comprised of drug reservoirs is deposited in specific tissue spaces where a therapeutic effect is required. The drug reservoirs are able to move freely throughout this tissue compartment to release biological agents which attract a variety of essential cells into the target tissue compartment. For use in tissue repair and regeneration, the drug reservoirs are designed to release attractants that cause homing of stem cells, accessory cells, and/or progenitor cells to the tissue compartment, with the progenitor cell and accessory cell depending on the specific tissue type. Some stem cells (including bone marrow hematopoietic and mesenchymal cells) may be attracted rather indiscriminately by reservoir depositings in a variety of different tissues, for example, liver, skeletal muscle, and brain. Exemplary progenitor cells include endothelial cells from local blood vessels, including both capillaries and larger arteries and veins, endothelial cell precursors from bone marrow, smooth muscle cells from the local tissue, circulating smooth muscle cell precursors, fibroblast precursors from the local tissue or from the blood, satellite cells from nearby muscle fibers, neural adult stem cells from specific regions of the cerebral cortex, neighboring myoblasts, and other cells. Accessory cells, including monocyte macrophages are attracted from the peripheral blood. Similar phagocytic accessory cells peculiar to specific tissues can be attracted from local sources, for example, Kupffer cells from the liver and microglia from the brain. For use in tumor regression, the drug reservoirs are designed to release attractants that cause homing of lymphocytes, macrophages, polymorphonuclear leucocytes, antigen-presenting cells, and/or natural killer cells. The presence of these cells in the tumor trigger a cascade of events that ultimately leads to tumor regression.

The reservoirs additionally contain for release biological agents that stimulate the attracted cells to do a variety of essential things, including to cause proliferation of extracellular matrix (ECM) materials, such as collagen and elastin, to cause degradation and remodeling of ECM, to synthesize mediators of cell adhesion, to synthesize intercellular signaling molecules, to differentiate into cells with specific functions (e.g., contractility and conduction for myocyte precursors, contractility for smooth muscle cells, ability to form specific kinds of cell junctions for endothelial cells, neurons, and striated muscle fibers). Through phased and multi-modal release of different biological agents the reservoirs can coordinate sequential stages of synthetic and differentiating activity by different cell populations.

The reservoirs will additionally release biological agents that influence the pattern of differentiation, function, quiescence, and/or apoptosis in critical cell populations. Release of suitable agents by the reservoirs promotes cell-based orchestration and harmonization of essential events in recovery of necrotic, or post-necrotic fibrosed regions to quasi-normal function.

### V. EXAMPLES

The following examples illustrate the preparation and use of compositions described herein and are in no way intended to limit the scope of the invention.

### Example 1

### Preparation of Polymeric Drug Reservoir

The following components were introduced into a reaction vessel: 12 grams of butyl methacrylate, 8 grams of ethylene glycol dimethacrylate, 0.2 grams of benzoyl peroxide dissolved in 80 grams of toluene, and 16 grams of polyvinyl alcohol dissolved in 400 grams of distilled water. The contents of the vessel were stirred by a mechanical stirrer to form a suspension of the organic phase in the aqueous phase. The stirring speed was increased to achieve an average droplet size of approximately 20 μm. Then, the mixture was heated to 70° C. and held at that temperature for three hours to complete the polymerization and the formation of the solid micro porous reservoir particles.

The solid particles were collected by filtration and washed several times with hot water to remove the suspending agent. The microporous particles were resuspended in water and steam was injected into the suspension to remove the toluene from the particles. After all of the toluene was removed the particles were washed with water, filtered, and dried. The solid microporous polymer microsphere particles were analyzed for particle size and porosity. The microporous reservoirs had an average particle size of 25 μm and an average particle porosity of 80%.

### Example 2

### Preparation of Composition with Biological Agents

The following cytokines and growth factors were loaded into the polymeric drug reservoirs prepared as described in Example 1. Lyophilized interleukin-12 (IL-12), granuolcyte macrophage colony stimulating factor (GM-CSF), interferon-gamma (IFN-γ), regulated on activation, normal T-cell expressed and secreted (RANTES), secondary lymphoid tissue chemokine (SLC), tumor necrosis factor-α (TNF-α), granulocyte colony stimulating factor (GCSF) and macrophage chemoattractant protein-1 (MCP-1) were loaded into the particles either as particles containing a single cytokine or growth factor or as particle reservoirs containing more than one cytokine or a combination of cytokine and growth factor. The desired agent or combination of agents were loaded into the by particles as follows. 1 mg of the reservoir particles were washed with neat isopropyl alcohol, filtered, and dried under vacuum. 100 μg of the lyophilized cytokine or growth factor was dissolved in buffered saline solution and the solution was filtered through a 0.2 μm filter. The filtered solution was mixed with the washed and filtered reservoir particles and stirred until the solution was adsorbed by the particles. The particles were lyophilized to remove the aqueous phase, leaving a lyophilized cytokine or growth factor entrapped in the reservoir particles.

To load combinations of cytokines, combinations of growth factors, or combinations of cytokines and growth factors a similar procedure was used. For example, 100 μg of lyophilized IL-12 and 100 pg of lyophilized GM-CSF were dissolved in buffered saline solution. The solution was filtered and mixed with washed and dried polymeric drug reservoirs until the solution was adsorbed. The particles were lyophilized leaving lyophilized IL-12 and GM-CSF entrapped in the microporous particles.

### Example 3

### Preparation Drug Reservoirs Loaded with GM-CSF and with G-CSF and RANTES

A. Particles with GM-CSF

100 μg of lyophilized GM-CSF was ultrasonically dispersed in 1 cc of a 4% methylene chloride solution of a 50/50 copolymer of (DL-lactide-co-glycolide). After uniform dispersion of the cytokine was achieved, the polymer solution containing the dispersed cytokine was poured into an excess of a precipitating solvent, such as petroleum ether. The spherical droplets of polymer solution containing dispersed GM-CSF were precipitated as solid spherical polymer particles containing GM-CSF. The solid particles were collected by filtration and lyophilized to remove residual solvent.

B. Particles with G-CSF and RANTES

Bioerodable microspheres containing combinations of cytokines, combinations of growth factors or combinations of cytokines and growth factors were prepared as follows. 100 μg of lyophilized G-CSF and 100 μg of lyophilized RANTES were ultrasonically dispersed in a 4% methylene chloride solution of a 50/50 copolymer of (DL-lactide-co-glycolide). After uniform dispersion of the cytokines was achieved, the polymer solution containing the dispersed cytokines was poured into an excess of a precipitating solvent such as petroleum ether. The spherical droplets of polymer solution containing dispersed G-CSF and RANTES were precipitated as solid spherical polymer particles containing G-CSF and RANTES. The solid particles were collected by filtration and lyophilized to remove residual solvent.

### Example 4

### In Vitro Release of IL-12 from Drug Reservoir Composition

Drug reservoir particles were loaded with IL-12 according to the procedure described in Example 2. The release of IL-12 from the biodegradable reservoirs was determined by incubating the particles in buffered saline solution and assaying for released IL-12 by enzyme-linked immunosorbent assays specific for IL-12. The release kinetics of IL-12 showed 60% of IL-12 was released over a period of 11 days.

### Example 5

### Composition for Cell and Tissue Regeneration

A major branch of the left anterior descending coronary artery of New Zealand White rabbits is ligated bringing about an infarct to the heart muscle served by the artery distal to the blockage of the artery. Two weeks after the infarct a bolus of microporous reservoirs and biodegradable microspheres containing GM-CSF, MCP-1, and RANTES, prepared as described in Example 3B, are delivered to the periphery of the infracted region via the femoral artery. The bolus of microporous reservoirs and biodegradable microspheres are delivered to the infracted area by the reservoir depositing device described in FIGS. 3 and 13. One group of animals receive reservoirs containing GM-CSF, MCP-1 and RANTES. Another group of animals serve as a control, receiving only blank reservoirs which contain no cytokine or growth factor. As a function of time, the extent of the arteriogenic response is measured by monitoring for an increase in number and size of collateral arteries on postmortem angiograms and by an increase of maximal blood flow during vasodilation. Cells isolated from treated animals are evaluated for the extent of monocyte infiltration into the region of the reservoirs and for the apoptosis (survival) of the attracted cells. In addition tissue is evaluated for the presence of pluripotent monocyte stem cells from the bone marrow via the circulation to the area of the reservoirs.

### Example 6

### In Vivo Release of IL-12 from Drug Reservoir Composition for Tumor Regression

Male and female BALB/c mice are each injected subcutaneously with 1×10⁶ viable CT-26 cells, a colon tumor cell line. The tumor cells are allowed to organize into a solid mass having the morphology and microstructure of a solid tumor. The tumor volume is measured every two days. Fourteen days after the establishment of a tumor mass the animals are divided into two groups. One group serves as a control group, the other group receives a single intratumoral injection of 150 μg of drug reservoirs containing IL-12. The tumor volumes of the control group and the group receiving the IL-12-loaded reservoirs are measured every four days. The decrease in tumor volume in the treated animals is observed.

Although the invention has been described with respect to particular embodiments, it will be apparent to those skilled in the art that various changes and modifications can be made without departing from the invention.

It is claimed:

1. A composition for inducing a cellular response, comprising
a first agent effective to attract one or more desired cells to a tissue site;
a second agent effective to stimulate activity of such cells; and
a third agent effective to influence survival of such cells.

2. The composition of claim 1, wherein said composition is for promoting regeneration of cells and/or tissue and said first agent is effective to attract one or more of stem cells, progenitor cells, and accessory cells to said tissue site.

3. The composition of claim 1, wherein said composition is for inducing an immune response to a tumor and said first agent is effective to attract one or more cells selected from the group consisting of T-lymphocytes, macrophages, polymorphonuclear leucocytes, antigen-presenting cells, and natural killer cells.

4. The composition of claim 1, wherein said second agent is effective to stimulate an activity selected from proliferation and differentiation.

5. The composition of claim 2, wherein said tissue is selected from the group consisting of skeletal muscle, liver, pancreas, brain, cardiac muscle, and central nervous system.

6. The composition of claim 5, wherein said tissue is cardiac tissue and said first agent attracts cells selected from the group consisting of circulating blood monocytes, circulating angiogenic cells, and circulating arteriogenic cells; said second agent stimulates release from said cells of factors to promote angiogenesis and/or arteriogenesis; and said third agent influences the survival of circulating blood monocyte-derived macrophages resident in the cardiac tissue.

7. The composition according to claim 6, wherein said first agent is selected from the group consisting of macrophage chemoattractant protein (MCP)-1, MCP-2, MCP-3, MCP-4, MCP-5, regulated upon activation, normal T-cell expressed and secreted cytokine (RANTES), Fraktalkines, macrophage inflammatory protein (MIP)-1-alpha, MIP-1-beta, N-farnesyl peptides, complement activation product C5a, leukotriene B4, platelet activating factor (PAF), transforming growth factor beta (TGF-beta), interleukins, granulocyte macrophage colony-stimulating factor (GM-CSF), granulocyte colony-stimulating factor (G-CSF), colony stimulating factor-1 (CSF-1), and macrophage colony-stimulating factor (M-CSF).

8. The composition according to claim 6, wherein said second agent is selected from the group consisting of macrophage chemoattractant protein (MCP)-1, MCP-2, MCP-3, MCP-4, MCP-5, tumor necrosis factor (TNF)-α, TNF-β, regulated upon activation, normal T-cell expressed and secreted cytokine (RANTES), Fraktalkines, macrophage inflammatory protein (MIP)-1-alpha, MIP-1-beta, N-farnesyl peptides, complement activating product C5a, leukotriene B4, platelet activating factor (PAF), transforming growth factor beta (TGF-beta), interleukins, granulocyte macrophage colony-stimulating factor (GM-CSF), granulocyte colony-stimulating factor (G-CSF), colony stimulating factor-1 (CSF-1), macrophage colony-stimulating factor (M-CSF), and lipopolysaccharide.

9. The composition according to claim 6, wherein said third agent is selected from the group consisting of GM-CSF, G-CSF, CSF-1, and M-CSF.

10. The composition according to claim 6, wherein one or more of said first, second, or third agents are selected from the group consisting of fibroblast growth factor (FGF, FGF-1, FGF-2), TGF-alpha, insulin-like growth factor (IGF-1), angiopoietin-1, angiopoietin-2, vascular endothelial growth factor (VEGF), constructs of VEGF such as VEGF-2, VEGF165, and VEGF121, platelet derived growth factor (PDGF)-A, PDGF-B, PDGF-BB, placental-derived growth factor (PIGF), and endothelial mitogenic growth factor.

11. The composition according to claim 3, wherein said first agent is selected from the group consisting of IL-8, MIG, IL-12, MCP-1, -2, -3, -4, -5, MIP-1 alpha, MIP-1 beta, MIP-1 gamma, and RANTES.

12. The composition according to claim 3, wherein said second agent is selected from the group consisting of GM-CSF and IL-12.

13. The composition according to claim 3, wherein said third agent is selected from the group consisting of MIG, platelet factor 4, MCP-1, -2, -3, and MIP-1 gamma.

14. The composition according to claim 1, wherein said agents are released from the composition simultaneously.

15. The composition according to claim 1, wherein said agents are released from the composition sequentially.

16. The composition according to claim 1, wherein said agents are packaged in a spherical drug reservoir comprised of a polymer.

17. The composition according to claim 16, wherein said drug reservoir has an external surface, and associated with said external surface, a biological ligand.

18. The composition according to claim 17, wherein said ligand is a cellular adhesion molecule.

19. The composition according to claim 16, wherein said composition is comprised of a plurality of drug reservoirs that are formulated into a dosage form selected from an emulsion, a gel, a paste, and a liquid.

20. A method for inducing a therapeutic response at a specific tissue site, comprising
depositing in or adjacent to a selected tissue site one or more drug reservoirs containing one or more therapeutic agents effective to
(i) attract to the tissue site one or more of stem cells selected from the group consisting of progenitor cells, accessory cells, T-lymphocytes, macrophages, polymorphonuclear leucocytes, antigen-presenting cells, and natural killer cells;
(ii) stimulate the one or more attracted cells to undergo an activity selected from proliferation and differentiation; and
(iii) influence the survival of said one or more attracted cells in said tissue.

21. The method of claim 20, wherein said tissue is cardiac tissue and said depositing is effective to promote arteriogenesis and/or angiogenesis.

22. The method according to claim 21, wherein said biological agents include (i) a first agent effective to attract cells selected from circulating blood monocytes, circulating angiogenic cells, and circulating arteriogenic cells; (ii) a second agent effective to stimulate release of factors to promote angiogenesis and/or arteriogenesis; and (iii) a third agent effective to influence the survival of circulating blood monocyte-derived macrophages resident in the cardiac tissue.

23. The method of claim 20, wherein said tissue is a tumor mass and said depositing is effective to attract cytotoxic cells into the tumor.

24. The method according to claim 20, wherein said depositing includes depositing one or more drug reservoirs into an interstitial tissue space.

25. The method according to claim 24, wherein said reservoirs are mobile within said interstitial tissue space.

26. A drug delivery device comprising:
a catheter shaft having a lumen extending therethrough to a port, a distal region being at least partly flexible, a proximal end being at least partly rigid, and a reinforced shaft segment at the proximal end;
at least one hypotube extending at least partly through the shaft proximal region;
a monorail segment positioned adjacent to the distal end of the catheter shaft and having a guidewire engagement segment;
a user interface having a distal end and a proximal end, and being rigidly connected at the interface distal end to the proximal end of the catheter shaft; and
a sheath connected to the proximal end of the interface for housing a movable piston.

27. The drug delivery device of claim 26, wherein said piston is movable along the length of the catheter shaft.

28. The drug delivery device of claim 26, further comprising a needle positioned at least partly in the catheter shaft distal region in a non-deployed state and deployable from the catheter shaft in a deployed state.

29. The drug delivery device of claim 28, wherein said needle is selected from the group consisting of a straight needle and a curved needle.

30. The drug delivery device of claim 26, further comprising a composite tube extending at least partly through the shaft distal region.

31. The drug delivery device of claim 30, wherein said composite tube contacts a proximal end of the needle.

32. The drug delivery device of claim 26, wherein said shaft distal and proximal regions are joined at a transition region.

33. The drug delivery device of claim 32, wherein said transition region is formed of a fused, thermoformed, thermoplastic elastomer spanning the proximal and distal regions.

34. The drug delivery device of claim 32, wherein said transition region includes an adaptor joined to a proximal end of the composite tube and to a distal end of at least one of the hypotubes.

35. The drug delivery device of claim 26, wherein said guidewire engagement segment further includes an endoluminal paving device for deposition of the composition along a wall of a vessel or a body cavity.

36. The drug delivery device of claim 35, wherein said catheter shaft port is positioned adjacent the endoluminal paving device to deliver the composition into the endoluminal paving device.

37. The drug delivery device of claim 36, said endoluminal paving device having an elongate, flexible cup with a central cavity for receiving the composition from the catheter shaft port.

38. The drug delivery device of claim 36, said endoluminal paving device having at least an upper opening and a lower opening for introduction of the guidewire through the cup.

39. The drug delivery device of claim 26, further comprising at least one balloon positioned on a distal portion of the monorail segment.

40. The drug delivery device of claim 39, wherein said at least one balloon comprises at least two balloons positioned on opposing sides of the monorail segment.

41. The drug delivery device of claim 39, wherein at least one of the at least one balloon has a biconical profile when inflated.

42. The drug delivery device of claim 26, further comprising:
one or more passages in the catheter shaft for introduction and removal of fluid.
